(19) United States
(12) Patent Application Publication
(10) Pub. No.: US 2015/0160236 A1
(43) Pub. Date: Jun. 11, 2015

(54) Methods For Determining Anti-TNF Therapeutic Response

(71) Applicant: New York Society For The Ruptured And Crippled Maintaining The Hospital For Special Surgery, New York, NY (US)

(72) Inventors: Mary K. Crow, New York, NY (US); Kleio Mavragani, New York, NY (US)

(21) Appl. No.: 14/557,208

(22) Filed: Dec. 1, 2014

Related U.S. Application Data
(63) Continuation of application No. 12/315,073, filed on Nov. 26, 2008, now abandoned.
(60) Provisional application No. 61/023,678, filed on Jan. 25, 2008.
(60) Provisional application No. 60/991,125, filed on Nov. 29, 2007.

(51) Int. Cl. G01N 33/68; C12Q 1/68

(52) U.S. Cl. CPC G01N 33/6866; C12Q 1/6876; G01N 33/6869; C12Q 2600/158; C12Q 2600/106; G01N 2333/56; G01N 2800/52; G01N 2333/565; G01N 2333/545

(57) ABSTRACT

The present invention relates to methods for identifying patients that will respond to treatment with anti-TNF-therapy, i.e., anti-TNF responder patients. In particular, the present invention relates to determining response to an inhibitor of tumor necrosis factor (TNF) in a patient with a chronic inflammatory disease by determining the activity of type I interferon in the patient. The invention further relates to quantification of type I interferon as a measure of predicting responsiveness to anti-TNF therapy in patients with a chronic inflammatory disease such as rheumatoid arthritis (RA), psoriatic arthritis, ankylosing spondylitis, juvenile chronic arthritis, lupus, Crohn's disease, as well as for cardiovascular disease.

### CROSS REFERENCE TO RELATED APPLICATIONS

This application claims priority from U.S. Provisional Application Ser. No. 60/991,125, filed Nov. 29, 2007 and U.S. Provisional Application Ser. No. 61/023,678, filed Jan. 25, 2008, each of which are hereby incorporated by reference in their entireties.

### FEDERALLY SPONSORED RESEARCH OR DEVELOPMENT

This invention was made in part in the course of research sponsored by the National Institutes of Health (NIH) Grant A1059893. The U.S. government may have certain rights in this invention.

### FIELD OF THE INVENTION

The present invention relates to methods for identifying patients that will respond to treatment with anti-TNF-therapy, i.e., anti-TNF responder patients.

### BACKGROUND OF THE INVENTION

Therapeutic blockade of tumor necrosis factor (TNF) represents a significant advance in the treatment of patients with chronic inflammatory diseases, particularly rheumatoid arthritis (RA), psoriatic arthritis, ankylosing spondylitis, juvenile chronic arthritis, as well as other inflammatory conditions such as Lupus, Crohn's disease, and others that might benefit from anti-TNF antibody treatment. In addition, TNF blockade may contribute to decreased cardiovascular morbidity, suggesting broader applications of this therapeutic approach to cardiovascular disease,

The development of biologic therapies that antagonize tumor necrosis factor-α (TNF) has substantially improved the extent and activity of inflammatory arthritis in many patients with rheumatoid arthritis (RA), resulting in decreased morbidity and mortality and clinically meaningful improvement in quality of life.

The two strategies for inhibiting TNF that have been most extensively studied to date consist of monoclonal anti-TNF antibodies and soluble TNF receptors (sTNF-R) (Table 1). Both constructs will theoretically bind to circulating TNF-α, thus limiting its ability to engage cell membrane TNF receptors and activate inflammatory pathways. Soluble TNF-R, but not anti-TNF antibodies, would also be expected to bind lymphotoxin. Alternative strategies include developing a TNF-α convertase enzyme (TACE) inhibitor in order to limit the amount of circulating soluble TNF.

The best studied of the monoclonal anti-TNF antibodies is infliximab (Remicade®), originally referred to as cA2. Infliximab is a chimeric human/mouse monoclonal anti-TNF-α antibody composed of the constant regions of human (Hu) IgG1κ, coupled to the Fv region of a high-affinity neutralizing murine anti-HuTNFα antibody. The antibody exhibits high affinity for recombinant and natural huTNF-α, and neutralizes TNF-mediated cytotoxicity and other functions in vitro. Because of the potential for an immune reaction to the mouse protein components of a chimeric antibody, an alternate strategy has been to develop a fully human anti-TNF monoclonal antibody. One such antibody, known as D2E7, also known as adalimumab, was generated by phage display technology. A high affinity murine anti-TNF monoclonal antibody was used as a template for guided selection, which involves complete replacement of the murine heavy and light chains with human counterparts and subsequent optimization of the antigen-binding affinity. D2E7 (Humira™) received FDA approval in December, 2002.

In the second approach to TNF inhibition, soluble TNF-R have been engineered as fusion proteins in which the extracellular ligand-binding portion of the huTNF-RI or huTNF-RII is coupled to a human immunoglobulin-like molecule. Although TNF-RI is thought to mediate most of the biological effects of TNF in vivo, engineered sTNF-RI and sTNF-RII constructs both appear to be effective in vivo inhibitors of TNF. Etanercept (sTNF-RII:Fc; Enbrel®) is the best studied of the sTNF-R and is approved for the treatment of rheumatoid arthritis in adults and in children. It is a dimeric construct in which two sTNF-RII (p75) are linked to the Fe portion of human IgG1. The dimeric receptor has a significantly higher affinity for TNF-α than the monomeric receptor (50-1000-fold higher), and the linkage to the Fe structure significantly prolongs the half-life of the construct in vivo. Although it also has an unnatural linkage site, anti-etanercept antibodies have been infrequent. Another mechanism for prolonging the half-life of monomeric receptors is via conjugation with polyethylene glycol. One such construct, PEG-sTNF-RJ (p55), has shown efficacy in several animal models of arthritis and is in clinical trials.

TABLE 1
TNF Inhibitors Currently Approved or in Development

| Name | Description | Status |
|---|---|---|
| Infliximab | Mouse-human chimeric anti-huTNF | FDA approved |
| Remicade ®) | mAb | |
| D2E7 | Fully human anti-huTNF mAb | FDA approved |
| (Humira ™) | | |
| Etanercept | p75sTNF-RII-Fc (dimeric) | FDA approved |
| (Enbrel ®) | | |
| (None) | PEG-p55sTNF-RI (monomeric) | In development |
| Lenercept | p55sTNF-RI-IgG1 (dimeric) | Development |
| | | terminated |

However, a considerable proportion of RA patients, ranging from 20 to 50% in clinical trials, demonstrate inadequate clinical or radiographic responses to these agents (1-12). Alternative approaches to therapy of RA and other inflammatory diseases have been developed, including agents that target B cells, such as rituximab (sold under the trade names Rituxan, MabThera and Reditux, a chimeric monoclonal antibody against the protein CD20, and agents that inhibit T cell activation, such as Orencia® (abatacept). Given the high cost and potential serious toxicities associated with anti-TNF agents, as well as the availability of alternative biologic therapies, identification of predictors of response to TNF antagonist therapy holds high significance for optimizing clinical management of RA patients.

Results from the British Society for Rheumatology Biologics Registry revealed that high levels of baseline disability as well as female sex are independent predictors of non-response to TNF antagonist therapy defined by the European League against Rheumatism (EULAR) criteria, while the concomitant use of methotrexate was a strong predictor of response at 6 months of therapy (13). Additional baseline predictors of remission have been reported including younger age, absence of rheumatoid factor (RF), lower disability levels as well as lower anti-cyclic citrullinated peptide antibody (anti-CCP) titers (14, 15), while high anti-CCP and RF titers but not the shared epitope or PTPN22 susceptibility alleles were associated with reduced response to anti-TNF treatment (16). Recent reports have suggested that higher levels of inflammation and TNF expression in the synovial membrane of RA patients prior to treatment are significant favorable predictors of response to anti-TNF therapy, but those measurements would be difficult to apply to clinical management of patients (17, 18). In addition, gene expression analysis of peripheral blood mononuclear cells (PBMC) from IRA patients treated either with infliximab or etanercept revealed a set of transcripts with potential predictive value (19, 20).

While TNF inhibition is of great therapeutic benefit and is increasingly considered a therapeutic option, not all patients demonstrate significant clinical responses to this therapeutic approach. Moreover, there are no data that indicate how to predict those who will and those who will not demonstrate a significant therapeutic response to TNF inhibition. It would be highly desirable to be able to select patients for anti-TNF therapy based on the likelihood that they will show improved disease activity after an adequate course of therapy (i.e., to identify responder patients). The goal of rational patient selection for anti-TNF therapy is particularly desirable because the cost of these therapies is high and a significant burden for patients and the healthcare system. In spite of the desirability of predicting therapeutic responses to TNF blockade, an approach to assess likelihood of improved disease activity in response to treatment with anti-TNF agents has not been validated or incorporated into medical care.

### SUMMARY OF THE INVENTION

The present invention provides methods for identifying patients that will respond to treatment with anti-TNF-therapy, i.e., anti-TNF responder patients.

In certain embodiments, the present invention provides methods for determining response to an inhibitor of tumor necrosis factor (TNF) in a patient with a chronic inflammatory disease comprising determining the activity or level of type I interferon in the patient.

In certain embodiments, the present invention provides a method for identifying a patient as a responder to anti-tumor necrosis factor (TNF) therapy comprising: a) contacting interferon responsive cells in vitro with a body fluid sample obtained from the patient, wherein the interferon responsive cells are not the patient's cells; b) detecting the expression level of at least one interferon-inducible gene (IFIG) by the interferon responsive cells of step a) thereby determining an interferon score, wherein the interferon score indicates responsiveness of the patient to anti-TNF therapy. In certain embodiments, the interferon score is a ratio of IFNβ/α and a ratio of IFNβ/α that is greater than about 0.8 identifies a patient as a responder to anti-(TNF) therapy.

In additional embodiments, the present invention provides a method for identifying a patient as a responder to anti-tumor necrosis factor (TNF) therapy comprising:
a) contacting interferon responsive cells in vitro with a body fluid sample obtained from the patient, wherein the interferon responsive cells are not the patient's cells;
b) detecting the expression level of at least one interferon-inducible gene (IFIG) by the interferon responsive cells of step a);
c) detecting the expression level of at least one interferon-inducible gene (IFIG) in a control sample by contacting interferon responsive cells in vitro with the control sample; and
d) comparing the expression level detected in step b) with the expression level detected in step c), wherein an increased expression level detected in step b) as compared with the expression level detected in step c) indicates that the patient will respond to anti-TNF therapy.

In additional embodiments, the present invention provides a method for identifying a patient as a responder to anti-tumor necrosis factor (TNF) therapy comprising determining the activity of type I interferon in the patient.

In additional embodiments, the activity or level of Type I IFN is used as a marker for determining responsiveness of RA patients to treatment with anti-TNF agents. This effect is independent of the type of anti-TNF agent used.

In additional embodiments, the present invention provides methods for quantification of type I interferon activity as a measure of predicting responsiveness to anti-TNF therapy in patients with other inflammatory conditions that might benefit from anti-TNF antibody treatment, such as Lupus and Crohn's disease.

In yet further embodiments, the present invention provides methods for quantification of type I interferon activity as a measure of predicting responsiveness to anti-TNF therapy in patients with a chronic inflammatory disease such as rheumatoid arthritis (RA), psoriatic arthritis, ankylosing spondylitis, juvenile chronic arthritis, as well as for cardiovascular disease. This quantification is independent of the type of anti-TNF agent used.

In additional embodiments, the present invention provides methods for diagnosing responsiveness to an inhibitor of tumor necrosis factor (TNF) in a patient with a chronic inflammatory disease comprising determining the activity of interferon-beta (IFN-β) compared to the activity of interferon-alpha (IFN-α) in the patient.

In additional embodiments, the present invention provides methods for diagnosing responsiveness to an inhibitor of tumor necrosis factor (TNF) in a patient with a chronic inflammatory disease comprising determining the activity of interferon-beta (IFN-β) in the patient.

In yet further embodiments, the present invention provides methods for diagnosing a rheumatoid arthritis patient comprising determining the activity of interferon-beta in the patient.

In yet additional embodiments, the present invention provides methods for diagnosing responsiveness to an inhibitor of tumor necrosis factor (TNF) in a patient with a chronic inflammatory disease comprising determining the expression of genes or proteins induced by interferon-beta (IFN-β) in the patient.

In yet additional embodiments, the present invention provides methods for diagnosing responsiveness to an inhibitor of tumor necrosis factor (TNF) in a patient with a chronic inflammatory disease comprising determining the expression of interleukin-1 receptor antagonist (IL-1ra).

In additional embodiments, the methods utilize a composite score including several parameters (two or more) described herein, including the IFN score and the IL-ra concentration. In yet additional embodiments, the composite score includes the IFN score plus the IL-ra concentration.

### BRIEF DESCRIPTION OF THE DRAWINGS

FIGS. 1A-B are plots showing disease activity [expressed as either disease activity score 28 (DAS28) or erythrocyte sedimentation rate (ESR)] at baseline (prior to anti-TNF therapy) and at visit 4 after initiating anti-TNF therapy (approximately 6.9±2 months) in RA patients with either high or low plasma type I interferon activity at baseline.

FIGS. 2A-B are plots showing both DAS28 and ESR at visit 4 (approximately 6.9±2 months) after initiation of anti-TNF therapy were significantly negatively correlated with baseline type I interferon activity.

FIG. 3 is a plot showing that the plasma type I interferon level was significantly higher at baseline in those patients who demonstrated an absolute change in DAS28 score from baseline to visit 4 (about 6.9 months after initiating anti-TNF therapy) greater than or equal to 1.9 than in those patients whose DAS 28 score changed less than 1.9 over the period of anti-TNF therapy.

FIG. 4 is a plot showing that RA samples exhibit inhibition by anti-IFN-α antibodies and by anti-IFN-β antibodies.

FIGS. 5A-B are plots showing baseline plasma IFN-β/IFN-α ratio and DAS28 at visit 1 and visit 4. FIG. 5B shows that samples having a relatively higher ratio of anti-IFN-βinhibition to anti-IFN-α inhibition at baseline identified those patients that had the best response to anti-TNF therapy, indicated by a lower disease activity score at visit 4 in these patients.

FIG. 6 shows IFN-β/IFN-α ratio in relation to DAS28 at visit 4.

FIG. 7 is a plot showing Type I interferon (IFN) plasma activity in patients with rheumatoid arthritis (RA) and healthy controls (HD). Plasma type I IFN activity was quantified using the WISH epithelial cell line. The dotted line shows the cut-off point between high and low levels defined as the type I IFN levels above the mean+2SD of a pool of healthy controls tested in the same assay. High levels of type I IFN were detected in 32% (16/50) of all RA patients at baseline compared to 6% (3/50) of a pool of healthy controls (p<0.0001).

FIGS. 8A-B are graphs showing the association of type I interferon plasma activity at baseline and visit 4 with response to anti-TNF treatment at visit 4 according to EULAR criteria. In FIG. 8A, a gradual increase of type I IFN levels was detected in poor, moderate and good responders (mean+SD 0.60±1.20 versus 1.53±2.30 versus 4.47±4.34, respectively, p=0.03). In FIG. 8B, a gradual increase of type I IFN plasma activity at visit 4 in the three responder groups is illustrated (mean±SD 0.23±0.01 versus 3.34±4.11 versus 8.53±3.12, p=0.01). p-values were determined by one-way ANOVA test.

FIGS. 9A-C are plots showing a significant inverse correlation between DAS28 at visit 2, 3, and 4 on the y axis and type I interferon activity level (called composite score on the graphs) in baseline serum.

FIGS. 10A-B are plots showing plasma type I IFN activity in anti-TNF and non anti-TNF treated RA patients at baseline and at follow up visits 4. A statistically significant increase in type I IFN plasma activity at visit 4 was seen in the RA group who received anti-TNF treatment compared to visit 1 (mean±SD 4.09±4.39 at visit 4 versus 1.83±2.84 at visit 1; p=0.04). No statistically significant difference in type I IFN plasma activity at visit 4 was observed in the RA non anti-TNF group compared to visit 1 (mean±SD 3.92±5.30 at visit 4 versus 2.40±3.82 at visit 1, p=0.92).

FIGS. 11 A-C are graphs showing the association of increased plasma IFNβ/α ratio with response to TNF antagonists and plasma interleukin-1 receptor antagonist (IL-1ra) level. DAS28 levels at baseline (FIG. 11A) and levels at visit 4 (FIG. 11B) in anti-TNF treated patients are demonstrated according to the baseline IFNβ/α ratio (the median value 0.8 of the distribution was used to differentiate two groups). FIG. 11C shows a statistically significant increase of IL-1ra levels, a protein known to be induced by interferon-beta, was found in the group of RA patients with baseline IFNβ/α ratio >0.8 compared to the group with baseline IFNβ/α ratio<0.8 (mean±SD 322.6±161.0 versus 185.8±68.72, p=0.04).

FIGS. 12A-C are plots showing an inverse relationship between IL-1ra levels at baseline and DAS28 levels at visit 2, 3, and 4 (significant with p=0.02 for visit 2 and 3 and p=0.05 for visit 4. IL-1ra level measured by ELISA is on x axis).

### DETAILED DESCRIPTION

The present invention provides methods for identifying patients that will respond to treatment with anti-TNF therapy, i.e., anti-TNF responder patients. In a particular embodiment, the invention provides a method for identifying rheumatoid arthritis patients that will respond to treatment with anti-TNF-therapy. Identifying patient responders to anti-TNF therapy is particularly desirable because the cost of these therapies is high and creates a significant burden for patients and the healthcare system. As alternative biologic therapies for rheumatoid arthritis are available, including those that target B cells or T cells, identifying individuals likely to be anti-TNF non-responders would allow selection of those alternative therapies.

The present invention relates to the discovery that quantification of type I interferon provides a measure that predicts improved disease activity in patients with RA after anti-TNF therapy. Measurement of type I interferon or its downstream molecular targets allows identification of those individuals who are less likely to respond to treatment with TNF inhibitors, as well as prediction of those individuals more likely to respond to those agents. Those individuals that are responsive to anti-TNF therapy are those with higher levels of type I interferon at baseline, as compared to those with lower levels of type I interferon. Those individuals exhibiting higher type I interferon or molecules induced by type I interferon at baseline are considered responsive to anti-TNF therapy, as shown in the data described herein.

Certain aspects of the invention are most directly applied to patients with RA, but may also be applicable to patients with other chronic inflammatory diseases such psoriatic arthritis, ankylosing spondylitis, juvenile chronic arthritis, as well as for cardiovascular disease. This quantification is independent of the type of anti-TNF agent used. Additionally, the invention also provides methods for quantification of type I interferon activity as a measure of predicting responsiveness to anti-TNF therapy in patients with other inflammatory conditions that might benefit from anti-TNF antibody treatment, such as Lupus and Crohns disease. Suitable anti-TNF agents include currently available agents (e.g., etanercept, infliximab or adalimumab) but also encompass additional therapeutic agents that inhibit TNF.

The measurement of type I interferon activity is accomplished by determining the capacity of serum, plasma or another body fluid to induce expression of interferon target genes (such as IFIT1, MX1, IFI44, PRKR, and many others) in type I interferon-responsive cell line cells (such as the WISH epithelial cell line cell). Suitable cells for the present in vitro assays include any suitable cells or cell lines that are IFN responsive, that is cells that are capable of binding to IFN in a manner reflective of the induction of interferon inducible genes. Examples of such cells include: WISH cells (American Type Culture Collection, reference CCL-25, Manassas, Va.), A-549 lung carcinoma cells (A-549 cells, ATCC reference CCL-185), G-361 human melanoma cells (G-361 cells, ATCC reference, CRL-1424), Hep-2 human laryngeal carcinoma cells (Hep-2 cells, ATCC reference CCL-23), human hepatocellular carcinoma cell line HepG2 (HepG2 cells, ATCC reference HB-8065), Huh-7 cell lines, AG1732 human cell line cells, primary human hepatocytes, and HeLa cells. Additionally, bovine kidney MDBK cells (MDBK cells, ATCC reference CCL-22), BT bovine cells (ATCC reference CRL-1390), guinea pig embryo cells (such as ATCC reference CCL-242), and SPEV pig cell line cells will be suitable in assays of the present invention. Many of these cell lines are described in the 2003/2004 Edition of the ATCC Catalog of Cell Lines & Hybridomas, American Type Culture Collection, Rockville, Md.

Type I interferon activity can be determined by measuring Type I interferon-inducible gene expression. Type I interferon-inducible gene expression is quantified by real-time polymerase chain reaction and a score is generated reflecting the relative level of expression of these genes. Type I interferon activity is also quantified based on the capacity of serum, plasma or other body fluids to activate a reporter gene construct that includes type I interferon responsive elements in association with a reporter read-out, such as luciferase.

Type I interferon activity can also be quantified based on measurement of one or more target gene products and proteins, such as CXCL10 (IP 10) which is highly produced in response to type I interferon. Non-limiting examples of these target gene products have been described and include Clorf29, myxovirus (influenza virus) resistance 1 (MX1), interferon-induced protein with tetratricopeptide repeats 1 (IFIT1), interferon-induced protein 44 (IFI44), protein kinase, interferon-inducible double stranded RNA dependent (PRKR), 2′-5′ oligoadenylate synthetase 3 (OAS3), guanylate nucleotide binding protein 1 (GBP1), human interferon regulatory factor-1 (IRF1), serpin peptidase inhibitor (SERPING1), chemokine (CXC motif) ligand 9 (CXCL9), chemokine (CXC motif) ligand 10 (CXCL10), proteasome subunit, beta type, 8 (PSMB8), proteasome subunit, beta type, 10 (PSMB10), G protein-coupled receptor 105 (GPR105), Fe fragment of IgG, high affinity Ia, receptor (CD64) (FCGR1A), interleukin-1 receptor antagonist (IL-1 ra, the IL-1ra gene is called IL-1RN and has GenBank Accession No. U65590), and soluble tumor necrosis factor receptor II (the TNF receptor II gene is called TNFRSF1B, with GenBank Accession No. NM_{—}001066). (For a listing of IFN inducible genes, See Crow, M. K. and Wohlgemuth, J. Microarray analysis of gene expression in lupus. Arthritis Res. Ther. 5:279-287, 2003; and Crow, M K., Kirou K. A., and Wohlgemuth J. Microarray analysis of interferon-regulated genes in SLE. Autoimmunity 36:481-490, 2003). It is expected that IFN-α and IFN-β are capable of inducing many, if not all of the above-listed gene products. (See, Der et al. Identification of genes differentially regulated by interferon α, β, or γ using oligonucleotide arrays, Proc. Natl. Acad. Sci. USA, 95:15623-15628, 1998). In particular, Der describes a number of candidate genes that may be preferentially induced by IFN-β including: hypoxia-inducible factor-1 (GenBank Accession No NM_{—}181054), Fra-1 (FOSL1; GenBank Accession No. AK 299050), and protein phosphatase 5 (PPP5C; GenBank Accession No. AK297095). Any one or more (e.g., two or more in combination) of these IFN induced genes or protein products are expected to be useful biomarkers in the methods of the present invention for identifying patients that will respond to treatment with anti-TNF therapy, i.e., anti-TNF responder patients.

Additionally, IFN-β has been reported to induce IL-1ra and soluble TNF receptor II. (See Molnarfi N., et al., The production of IL-1 receptor antagonist in IFN-β-stimulated human monocytes depends on the activation of phosphatidylinositol 3-kinase but not of STAT1. J. Immunol. 174:2974-2980, 2005; and Comabella M., et al., Induction of serum soluble tumor necrosis factor receptor H (snTNF-RII) and interleukin-1 receptor antagonist (IL-1ra) by interferon beta1b in patients with progressive multiple sclerosis. J. Neural. 2008, May 20 [Epub ahead of print).

Additionally, certain enzyme-linked immunosorbant assays (ELISAs) provide an accurate quantification of type interferon activity. Any assay that accurately quantifies type I interferon activity may be effective in predicting who will and who will not respond to anti-TNF therapy in the methods of the present invention.

Additionally, type I interferon activity can be measured by using antibodies against any of the type I interferon family members, Type I interferon encompasses a large family of gene products including 13-14 interferon-alphas (IFN-α), one IFN-13, one interferon omega (IFN-Ω), and others (See Pitha, P. S. and Kunzi, P. M., Curr. Top. Microbiol. Immunol., 2007; 316:41-70). Thus, type I interferon activity can be detected using anti-IFN-α, anti-IFN-β, anti-IFN-Ω, or any antibodies, or antibody fragments reactive with any of the type I interferon gene products. A listing of type I interferon gene products is shown in Table 2 along with GenBank Accession numbers for the coding sequences and protein products.

TABLE 2
GenBank References

| | |
|---|---|
| IFNβ1 | NM_002176.2→NP_002167.1 interferon, beta 1, fibroblast |
| IFNΩ1 | NM_002177.1→NP_002168.1 interferon, omega 1 |
| IFNα21 | NM_002175.2→NP_002166.2 interferon, alpha 21 |
| IFNα4 | NM_021068.2→NP_066546.1 interferon, alpha 4 |
| IFNα7 | NM_021057.2→NP_066401.2 interferon, alpha 7 |
| IFNα10 | NM_002171.1→NP_002162.1 interferon, alpha 10 |
| IFNα16 | NM_002173.2→NP_002164.1 interferon, alpha 16 |
| IFNα17 | NM_021268.1→NP_067091.1 interferon, alpha 17 |
| IFNα14 | NM_002172.2→NP_002163.2 interferon, alpha 14 |
| IFNα5 | NM_002169.1→NP_002160.1 interferon, alpha 5 |
| IFNα6 | NM_021002.1→NP_066282.1 interferon, alpha 6 |
| IFNα13 | NM_006900.3→NP_008831.3 interferon, alpha 13 precursor |
| IFNα2 | NM_000605.3→NP_000596.2 interferon, alpha 2 |
| IFNα8 | NM_002170.3→NP_002161.2 interferon, alpha 8 |
| IFNα1 | NM_024013.1→NP_076918.1 interferon, alpha 1 |
| IFNε1 | NM_176891.3→NP_795372.1 interferon epsilon 1 |
| IFNκ | NM_020124.2→NP_064509.2 interferon kappa precursor |

In certain embodiments, detection of a subtype of Type I interferon, interferon beta (IFN-β) is utilized as a serum biomarker for RA. This determination is surprising in view of previous characterizations of Type I interferon subtypes for SLE and Sjogren's syndrome. It was previously reported in SLE and Sjogren's syndrome that nearly all of the type I interferon activity that can be measured in serum or plasma can be inhibited by anti-IFN-α antibody. Thus, the majority of type I interferon activity that can be measured in serum or plasma in SLE and Sjogren's syndrome patients is attributable to IFN-α. Thus, certain embodiments of the invention provide a diagnostic tool for identifying RA patients by detecting IFN-β as a serum or plasma biomarker for RA.

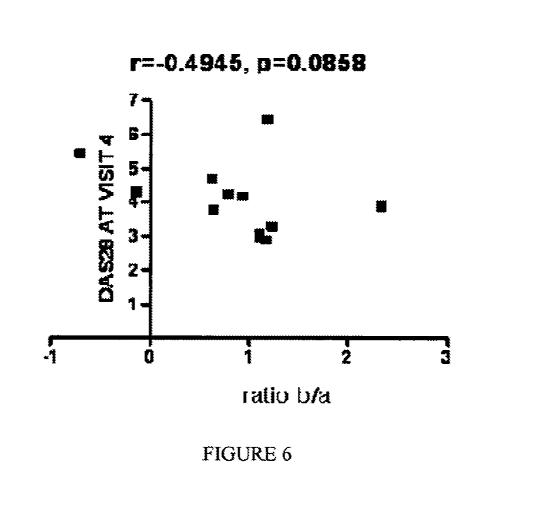

Additionally, detection of IFN-β when used in combination with detection of IFN-α in serum or plasma samples, using anti-IFN-β and anti-IFN-α antibodies or any other suitable method including suitable antibody fragments, provides a method for identifying patients who will be responsive to anti-TNF therapy. It is also contemplated that an amount or range of IFN-β predictive for anti-TNF responders can be developed based upon a suitable sample size of results using data of ratios of IFN-β to IFN-α (e.g., FIG. 6). Thus, certain embodiments of the invention provide a diagnostic tool for identifying patients who will be responders to anti-TNF therapy by utilizing the ratio of IFN-β to IFN-α as a serum biomarker for responder patients.

A “control”, “control value” or “reference value” in an assay is a value used to detect an alteration in, e.g., transcriptional activity of a gene, levels of a protein or mRNA detected in a sample taken from a patient or measured in a reconstituted system, or any other assays described herein. For instance, the presence of mediators in a patient sample, such as serum or plasma that stimulate expression of interferon-inducible genes in IFN responsive cells such as WISH cells, can be tested or verified by measuring the levels of RNA in the WISH cell followed by RT-PCR and real time PCR and comparing the results to a control WISH cell that has not been exposed or contacted with the patient sample. In addition, modulation, i.e., up- or down-regulation of the transcriptional activity of an agent (e.g., a test compound) can be determined using a WISH cell exposed to a patient sample by comparing the measured value of transcriptional activity from the patient sample to that of a control value. The control or reference value may be, e.g., a predetermined reference value, or may be determined experimentally. For example, in such an assay, a control or reference may be, e.g., the transcriptional activity of a gene in the absence of an agent or test compound (for comparison with transcriptional activity in the presence of the agent); or any other suitable control or reference. In a diagnostic assay, a reference or control value may be obtained by comparing e.g., a nucleotide sequence, or a nucleotide or protein level measured, in a sample taken from a patient predisposed to or suspected of suffering from, a disease, to a corresponding sequence or measured value of a sample taken from a healthy, or “control” individual.

A “sample” refers to a biological material which can be tested for the presence of mediators that stimulate the expression of interferon-inducible genes. Such samples can be obtained from subjects, such as humans and non-human animals, and include blood and blood products, synovial fluid, tissue, especially glands, biopsies, plural effusions, cerebrospinal fluid (CSF), ascites fluid, and cell culture.

Cells, tissues, and fluids useful in the methods of this invention include whole blood, plasma, serum, urine, nasal secretions, synovial fluid, cerebrospinal fluid, ocular secretions, vaginal secretions, and saliva. In certain embodiments blood, plasma, or serum is utilized in the practice of this invention.

When the sample is blood, methods may include processing the blood by a means known to the art, such as filtration or centrifugation, for separating plasma or serum which is to be assayed. As used herein, “whole blood” refers to blood as drawn. Whole blood contains a substantial amount of cells. As used herein, “plasma” refers to blood with no substantial amount of cells. Plasma does contain clotting factors. As used herein, “serum” refers to blood without a substantial amount of cells or clotting factors.

The term “cDNA” refers to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred.

In accordance with the present invention there may be employed conventional molecular biology, microbiology, cell biology, and recombinant DNA techniques within the skill of the art. Such techniques are explained fully in the literature. See e.g. Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Freshney (2005) Culture of Animal Cells: A Manual of Basic Technique. Wiley-Liss; 5 edition (Jul. 29, 2005); Coligan et al. eds, (2005) Current Protocols in Immunology, John Wiley and Sons, Inc.: Hoboken, N.J.,

In certain embodiments, the present invention may utilize DNA segments that are complementary, or essentially complementary, to the sequences described herein. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a specified nucleic acid segment, under relatively stringent conditions such as those described herein below. Such sequences may encode a complete protein product or a functional or non-functional fragment thereof.

Similarly, any reference to a nucleic acid may be read as encompassing a host cell containing that nucleic acid and, in some cases, capable of expressing the product of that nucleic acid. In addition to therapeutic considerations, cells expressing nucleic acids may prove useful in the context of screening for agents that induce, repress, inhibit, augment, interfere with, block, abrogate, stimulate or enhance the expression or detectability of IFN transcripts, IFN polypeptides, or desired fragments of IFN transcripts or IFN polypeptides.

Hybridizing nucleic acid segments may be relatively short nucleic acids, often termed oligonucleotides. Sequences of at least 10 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides are contemplated as well. Such oligonucleotides will find use, for example, as probes in Southern and Northern blots and as primers in amplification reactions.

Suitable nucleic acid hybridization conditions will be well known to those of skill in the art. Accordingly, the nucleotide sequences of the invention may be used for their ability to selectively form duplex molecules with complementary stretches of DNA fragments. Depending on the application envisioned, one will desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by about 0.02 M to about 0.15 M NaCl at temperatures of about 50° C. to about 70° C. Such selective conditions tolerate little, if any, mismatch between the probe and the template or target strand, and would be particularly suitable for isolating crystal protein-encoding DNA segments. Detection of DNA segments via hybridization is well-known to those of skill in the art, and the teachings of U.S. Pat. Nos. 4,965,188 and 5,176,995 are exemplary of the methods of hybridization analyses.

In certain applications, for example, substitution of amino acids by site-directed mutagenesis, it is appreciated that lower stringency conditions are required. Under these conditions, hybridization may occur even though the sequences of probe and target strand are not perfectly complementary, with mismatches at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions include utilizing approximately 10 mM Tris-HCl (pH 8.3), 50 mM KCl, 1.5 μM MgCl₂, at temperatures ranging from approximately 40° C. to about 72° C. Formamide and sodium dodecyl sulfate (SDS) also may be used to alter the hybridization conditions.

In certain embodiments, it will be advantageous to employ nucleic acid sequences (e.g. primers or probes) utilized in the present invention in combination with an appropriate means, such as a label, for determining hybridization. A wide variety of appropriate indicator means are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmental undesirable reagents. In the case of enzyme tags, colorimetric indicator substrates are known that can be employed to provide a means visible to the human eye or spectrophotometrically, to identify specific hybridization with complementary nucleic acid-containing samples.

In general, it is envisioned that the hybridization probes described herein will be useful both as reagents in solution hybridization as well as in embodiments employing a solid phase. In embodiments involving a solid phase, the test DNA (or RNA) is adsorbed or otherwise affixed to a selected matrix or surface. This fixed, single-stranded nucleic acid is then subjected to specific hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface so as to remove nonspecifically bound probe molecules, specific hybridization is detected, or even quantitated, by means of the label.

### EXAMPLES

The following examples are included to demonstrate certain embodiments of the invention. It should be appreciated by those a skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Patients and Methods

Fifty RA patients from a single center (38 anti-TNF-treated RA patients and 12 treated with other agents) were studied. Type I IFN activity was measured using a reporter cell assay. Disease status at baseline and follow-up visits was assessed using the Disease Activity Score (DAS28). The response was classified according to the European League against Rheumatism (EULAR) improvement criteria.

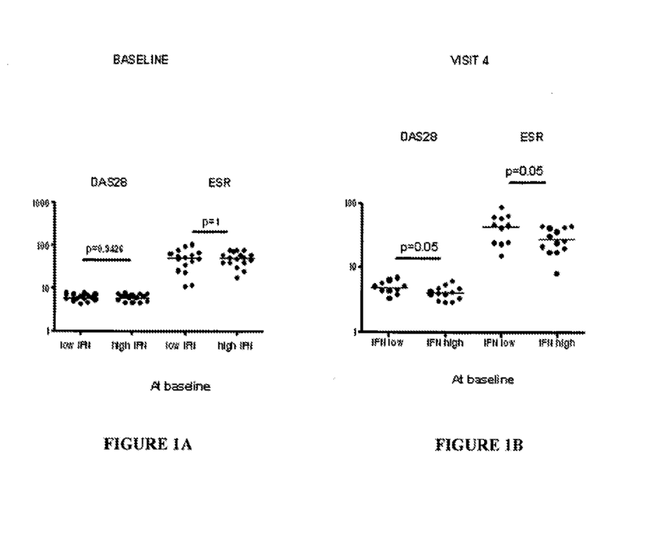

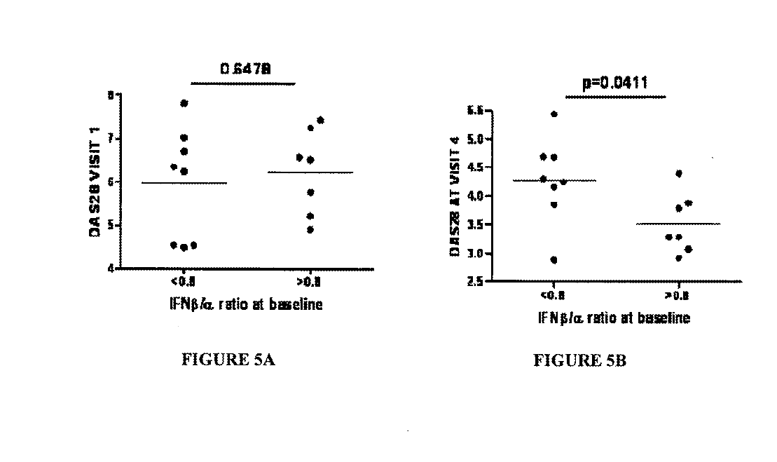

For the results shown in FIGS. 1-12, the study population included three subject cohorts: 1) RA patients receiving care at the Los Angeles County+University of Southern California Medical Center Rheumatology clinics (n=38) who, based on the clinical judgment of their attending physicians, required the addition of a TNF antagonist [anti-TNF group, (etanercept, n=16; infliximab, i=9; adalimumab, n=13)] to their therapeutic regimens and who agreed to such treatment; 2) RA patients receiving medical care at the same clinics (total n=12) who either refused (n=2) or were felt not to be candidates for TNF antagonist therapy (n=10) [non-anti-TNF group]; and 3) healthy volunteers (n=50) of similar age and sex distribution with the RA cohort. Baseline characteristics of the RA cohorts have been previously described (32).

Outcome at visit 4 was categorized according to DAS scores, defined by the EULAR improvement criteria (33, 34), into three groups: no response, moderate response and good response, based on the DAS28 at visit 4 and absolute change in DAS28 from baseline. The definition of a good responder was an improvement of at least 1.2 U and an absolute score of ≦3.2 U, while the definition of a non-responder was an improvement of <0.6 U and an absolute score of >5.1 U. Moderate responses fall in between. Those patients who discontinued their TNF antagonist therapy prior to the end of the follow-up at visit 4 were labeled as non-responders.

### IFN Assay: Plasma Type I IFN Activity.

Type I IFN activity was detected using a reporter cell assay according to described methods (35-37, and also in co-pending U.S. application Ser. No. 11/431,775). In brief, cells of the WISH epithelial cell line (ATCC #CCL-25, Manassas, Va.) express the type I IFN receptor and are highly responsive to type I IFN. WISH cells were plated at a density of 5×10⁵ cells/ml in 96 well plates in Minimum Essential Media (Celigro, Herndon, Va.) with 10% fetal calf serum (FCS) and then were cultured with 50% patient plasma for 6 hours. Recombinant human IFNα (IFNaA, Biosource International, Camarillo, Calif.) and media only were used as positive and negative controls, respectively.

Preparation of cDNA-Quantitative Real-Time Polymerase Chain Reaction.

Total cellular mRNA was purified from stimulated cells at the end of the culture period using the Qiagen Turbocapture oligo-dT coated 96 well plate system as per the manufacturer's protocol (Qiagen, Valencia, Calif.), Total cellular mRNA was reverse-transcribed to cDNA immediately following purification using the TaqMan Reverse Transcription Reagents (Applied Biosystems, Foster City, Calif.).

Quantitative real-time polymerase chain reaction (PCR) was used to quantify specific cDNAs using the Bio-Rad SYBR Green intercalating fluorophore system with a Bio-Rad 1-cycler thermocycler and fluorescence detector (Bio-Rad, Hercules, Calif.). Primers for genes that are highly induced by type I IFN signaling were used in the PCR reaction on the WISH cell-derived cDNAs (Operon, Huntsville, Ala.): interferon induced with tetratricopeptide repeats 1 ((SEQ ID NO:1) IFIT-1, Forward CTCCTTGGGTTCGTCTATAAATTG; (SEQ ID NO:2) Reverse AGTCAGCAGCCAGTCTCAG), double-stranded RNA-dependent protein kinase (PKR) (SEQ ID NO:3) (Forward CTTCCATCTGACTCAGGTTT; (SEQ ID NO:4) Reverse TGCTTCTGACGGTATGTATTA) and myxovirus resistance 1 (MX-1) (SEQ ID NO:5) (Forward TACCAGGACTACGAGATTG; (SEQ ID NO:6) Reverse TGCCAGGAAGGTCTATTAG). The housekeeping gene glyceraldehyde-3-phosphate dehydrogenase (GAPDH, (SEQ ID NO:7) Forward CAACGGATTTGGTCGTATT; (SEQ ID NO:8) Reverse GATGGCAACAATATCCACTT) was also quantified in the cDNA samples to control for background gene expression. Threshold values were recorded for each sample at the logarithmic portion of the amplification. Melt curve analysis was used to ensure the specificity of the PCR product. The type I IFN-induced genes were compared with housekeeping gene expression to determine relative expression. The relative expression was then normalized to the relative expression of the respective genes in unstimulated cells from the same population. RA samples were compared to the mean and SD of a pool of healthy donors in the same assay and the sum of the number of SD above healthy donors for each of the three genes was calculated for each sample.

Real-time PCR following reverse transcriptase polymerase chain reaction (RT-PCR) as utilized in the present methods is also described in co-pending U.S. application Ser. No. 11/431,775. For these reactions, RNA was extracted from each cell lysate using the Qiagen Turbocapture oligo-dT coated 96 well plate system as per the manufacturer's instructions (Qiagen, Valencia, Calif.). An aliquot of 0.4 μg of this RNA was reverse-transcribed to cDNA in a 20 μl reaction using SuperScript III RNase H-Reverse Transcriptase (Invitrogen, Carlsbad, Calif.). Alternatively, TaqMan Reverse Transcription Reagents were used (Applied Biosystems, Foster City, Calif.). cDNA obtained from each sample was diluted 1:40, and 10 μl was amplified in a 25 μl real-time PCR reaction using 0.4 μM of sense and antisense primers and the 2× iQ SYBR Green intercalating fluorophore system (Bio-Rad, Hercules, Calif.). Glyceraldehyde-3-phosphate dehydrogenase (GAPDH) was used as a housekeeping gene control. Primers for all target genes and GAPDH were designed using Beacon Designer 2.06 software (Premier Biosoft International, Palo Alto, Calif.) in conjunction with the DNA mfold 3.1 program to exclude sequences with significant secondary structure (See, Zuker M. Mfold web server for nucleic acid folding and hybridization prediction. Nucleic Acids Res. 2003; 31:3406-3415).

WISH cells cultured with medium were included as a control in each assay to provide a basis for normalization across experiments. Results for each experimental culture condition are expressed as relative expression (RE) compared to WISH cells cultured with medium.

Experiments may combine real-time, kinetic RT-PCR detection together with an internal homologous control that can be simultaneously detected alongside the target sequences. (See, for discussion of quantitative RT-PCR: Freeman W M, Walker S J, Vrana K E, Quantitative RT-PCR: pitfalls and potential. BioTechniques, 1999; 26(1):112-125; See also: Bustin S A. Absolute quantification of mRNA using real-time reverse transcription polymerase chain reaction assays. J Mol Endocrinol 25:169-93, 2000; and Wong M L, Medrano J F. Real-time PCR for mRNA quantitation. Biotechniques 39:75-85, 2005).

The advantages of real-time PCR include that it is quantitative, that only a small number of cells are required to generate sufficient cDNA for analysis, and the assay is simple to perform once the conditions for detecting a specific transcript are established.

The real-time quantitative amplification of gene expression and analysis is as described in Kirou et al. (Kirou K A, Lee C, George S, Louca K, Papagiannis I G, Peterson M G, et al. Coordinate overexpression of interferon-alpha-induced genes in systemic lupus erythematosus. Arthritis Rheum 2004; 50(12):3958-67, and 30—Kirou K A, Lee C, Crow M K. Measurement of cytokines in autoimmune disease. Methods Mol Med 2004; 102:129-54.) and is described generally as follows. Real-time PCR is performed using the Bio-Rad iCycle IQ Real-Time Detection System (Bio-Rad, Hercules, Calif.), although numerous other cyclers are available and suitable for use in the present methods. Quantitation is achieved by measuring the increase in fluorescence during the exponential phase of the amplification, which is in real time. An excitation system via an excitation wheel directs the light onto all of the wells of a 96-well plate. Next, fluorescent molecules from each well emit fluorescent light through an emission filter wheel and an image intensifier; this light is eventually detected by the CCD camera. The data are then transferred to a computer and analyzed with Bio-Rad software.

There are at least four methods used for real-time PCR: molecular beacons, hydrolysis probes (e.g. TaqMan), hybridization probes, and DNA-binding dyes (e.g. SYBR Green I), with any of these methods being suitable for steps in the present methods. The present results were obtained using the SYBR Green I method using the Bio-Rad iCycle IQ Real-Time Detection System with the target and housekeeping genes amplified in separate wells from the same template and by running a melt curve analysis of the PCR products at the end of the reaction.

cDNA standards are prepared for each gene to be amplified. Plasmids are typically used, but any known sample with a high expression for the desired gene can be used. For example, the cDNA standard for IFN-induced protein with tetratricopeptide repeats 1 (IFIT1) was made from a sample of 2 million PBMCs stimulated with 1000 U/mL of IFNα for 24 hours. Multiple aliquots of cDNA samples are made and frozen. Prior to PCR, cDNA dilutions for standard curves are made, typically by making three 10-fold dilutions in duplicate per plates. In later assays testing the performance of the reaction, typically only two dilutions are made. The 1:10 dilutions of all test/unknown cDNA samples are made and 10 μL (for more accurate pipetting) of each sample is added, in duplicate to wells in the 96-well PCR plate.

The Supermix for each primer is prepared as follows: (N+2)×12.5 μL of Bio-Rad SYBR Green Mix; (N+2) μL of the S and AS primer solution; and (N+2)×1.5 μL of DEPC-treated water. Next, 15 μL of the Supermix is added to each well. The final volume per reaction is 25 μL, with concentrations of 0.4 μM for each primer, of 25 U/mL of iTac DNA polymerase, of 0.2 mM each of dNTPs, and of 3 mM of Mg⁺⁺. The Supermix also contains SYBR Green I, a DNA intercalating dye responsible for the fluorescence emission during the reaction used to determine the starting quantity of the gene in question. As the cDNA is amplified during the successive reaction cycles, more and more dye binds to DNA, and more fluorescence is emitted. After the Supermix is added to each well, the plate is sealed with optical tape. Next, the plate is spun in a centrifuge at 300 g, at 4° C. for 3-5 minutes. The plate is covered with foil and kept refrigerated at 4° C. until it is placed into the machine. The time between preparation and run on the machine is minimized.

The PCR reaction is run according to manufacturer's instructions by placing the 96-well plate in the PCR machine and opening the iCycler software. The software is initiated by selecting the Library window and the View Protocol tab to select the most suitable protocol for the reaction. Typically, the cycle chosen is 2StepAmp+Melt.tmo, which includes five cycles. Cycle 1 consists of 1 repeat and 1 step with dwell time of 3 minutes (min.) at 95° C. Cycle 2 consists of 40 repeats of step 1 (10 second (sec) dwell time at 95° C.) and step 2 (45 sec of dwell time at 55° C.). Cycles 3 and 4 each have 1 repeat and 1 step, with 1 min. dwell times at 95° C. and 55° C., respectively. Finally, cycle 5 consists of 80 repeats of step 1 with a 10 sec dwell time starting at 55° C. and positive increments of 0.5° C. for every repeat. By clicking on Edit This Protocol, the settings of the various cycles may be changed according to the experimental conditions. The methods used for analyzing the IFIGs of the present conditions used the above cycles with the annealing temperature of 58° C.

Two genes are amplified: a target gene and a housekeeping gene, which serves as the internal control for each sample. GAPDH is the housekeeping gene used in the present experiments (previously described and available at GenBank Accession No. NM_{—}002046.3). The specificity of the PCR reactions for the amplified genes is confirmed by examining the melting curve (generated during cycle 5 of the reaction) for each gene amplification.

The data is analyzed using Bio-Rad iCycle iQ software (version 3.0a). Optionally, this analysis can be done on a personal computer away from the PCR machine using the appropriate software. The View Post Run Data tab is selected, the experiment file is selected, and then Analyze Data is selected to obtain results. The amplification curves from all wells are displayed on the Data Analysis window under the PCR Quantification tab (the x-axis represents the PCR cycle and the y-axis represents the fluorescence intensity). The melting curves and the standard curves can also be viewed by clicking on the corresponding tabs.

To assess the expression of each gene for all unknown samples, the corresponding wells for that gene (unknowns and standards) are selected, the Select Wells box is selected (highlighting the relevant wells), and Analyze Selected Wells is clicked. The program then automatically sets the best possible threshold line that will horizontally intersect all amplification curves at their exponential phase. This can be best visualized when Analysis Mode is set at the PCR Baseline Cycle Subtracted option and with the graph adjusted so that the fluorescence intensity axis is expressed in a Log scale. The threshold line will determine the threshold cycle of amplification for each sample and will plot it (y-axis) against the Log Starting Quantity of the particular gene (x-axis). The latter value is known for the standards (and typically is set a 1, 10, or 100 according to their dilutions) and is calculated for the unknowns in the standard curve view of the results. The standard curve also gives the correlation coefficient (optimally above 0.98) and the PCR efficiency (optimally between 80 and 120%).

The curves typically show only one melting peak for each amplified gene. If more than one peak is present, a primer dimer (melt peak lower than amplicon peak) may have formed, or another gene may have been concurrently amplified. Either of these situations is undesirable and the results would be discarded.

The threshold cycle values for the target and reference genes for each sample are entered into an Excel file and subtracted from the corresponding values of a reference sample (usually the one with no stimulation). These differences are then used as exponents with base the sum of the efficiency of the PCR reaction expressed as a decimal plus 1 (i.e., for efficiency of 98%, this would be 1.98). By definition, therefore, the corresponding differences of the reference sample for both the target and housekeeping genes will be 0, which when used as exponents will result in values of 1. Finally, the target gene values are divided by the housekeeping gene values for each sample, and the result is the relative expression value for each unknown sample.

In an example of determining IFIT1 target gene expression, PBMCs from a healthy donor were either left untreated or were cultured with 200 U/ml, IFNα. Two of the IFNα treated samples were also treated with anti-IFNα Ab at a concentration of 200 neutralization units/mL or an isotype control Ab. Cell lysis, RNA isolation, reverse transcription, and real-time PCR amplification followed for IFIT1 and HPRT1 (used in this example as a housekeeping gene). The amplification, melt, and standard curves for each gene were determined as described above. The efficiency for each reaction was determined as 111.4% or E=1.114 for IFIT1 and 96.8% or E=0.968 for HPRT1. The threshold cycles (Ct) for each sample and for each gene were calculated. The Δ Ct or differences of the Ct of each cycle, from the Ct of medium (reference sample) for the two genes were determined. The E+1 for each gene (2.114 and 1.968) was also calculated as raised to the corresponding Δ Ct power for each sample (E+1)^{Δ Ct}. The relative expression (RE) of IFIT1 for the sample was determined by dividing its (E+1)^{≢ Ct} value by the (E+1)^{Δ Ct} value of HPRT1.

The mean (M) and SD of each IFIG for the group of healthy donors (M_{HD} and SD_{HD}) were used to calculate that gene's expression score for each study subject, defined as the number of SD_{HD} above the M_{HD}. Cumulative IFN scores, representing the sum of scores for each of the tested genes preferentially induced by the particular IFN were derived from each subject. An IFNα score was considered high if it fulfilled the following criteria: the IFN score of the study subject is greater than the mean plus 2 standard deviations of the IFN scores of a group of healthy control subjects.

Statistical Analysis.

Comparison between continuous variables was performed by the paired t-test for matched data and by the unpaired t-test or the Mann-Whitney test for normally and not-normally distributed non-matched data, respectively. Comparisons between categorical variables were performed by Fisher's exact two sided tests. Comparisons between three groups were performed using the one-way ANOVA test for normally distributed data. Correlations between continuous variables were determined using the nonparametric Spearman's test.

### Determination of IFNβ/α Ratio

To test whether the ability of RA plasma to stimulate expression of type I IFN-inducible genes is predominantly due to the presence of IFNα or IFNβ, a subset of available samples of anti-TNF treated RA patients with detectable type I IFN activity at baseline was incubated with monoclonal anti-IFNα or anti-IFN antibody (10 μg/ml) (Chemicon, Temecula, Calif.). These antibodies are known to be specific for the designated IFNs. An IFNβ/α ratio for each sample tested was then calculated based on the ratio of % inhibition of type I IFN activity by anti-IFNβ to the % inhibition of type I IFN activity by anti-IFNα antibody.

### IL-1 Receptor Antagonist (Il-1ra) ELISA

Determination of IL-1ra levels was performed by a solid-phase ELISA according to manufacturer's instructions (Quantikine H S, R&D Systems, Minneapolis, US). This assay employed the quantitative “sandwich” enzyme immunoassay technique.

### Statistical Analysis

Comparison between continuous variables was performed by unpaired t-test or the Mann-Whitney test for normally and not normally distributed data respectively. Comparisons between categorical variables were performed by Fisher's exact two sided tests. Comparisons between three groups were performed using the one-way ANOVA test for normally distributed data. Correlations between continuous variables were determined using the nonparametric Spearman's test. Mean values between RA groups (treated with an anti-TNF agent or DMARD) prior to and after therapy were compared using paired t-tests.

Other methods such as luciferase reporter assays may also be used in the detection of the expression of one or more interferon-inducible genes. Luciferase reporter assays are well-known in the art (See for example, Luciferase Assay System (Promega, Madison, Wis.).

### Example 1

Blood samples were obtained from RA patients at baseline or approximately 6 months after beginning therapy with an anti-TNF agent (e.g., etanercept, infliximab or adalimumab). Type I interferon activity was measured using an assay that quantifies type I interferon activity in plasma. WISH epithelial cell line cells were incubated with patient plasma for 6 hours and expression of several interferon-inducible genes was measured by real-time PCR. An interferon score was derived using these data.

FIGS. 1A-B demonstrate disease activity [expressed as either disease activity score 28 (DAS28) (FIG. 1A) or erythrocyte sedimentation rate (ESR)] at baseline (prior to anti-TNF therapy) and at visit 4 after initiating anti-TNF therapy (FIG. 1B) (approximately the six month time point) in RA patients with either high or low plasma type I interferon activity at baseline. DAS28 and ESR were comparable among RA patients with high and low plasma type I interferon activity at baseline, but after six months of therapy those patients with high type I interferon activity showed lower DAS28 and ESR than did those with low type I interferon activity at baseline.

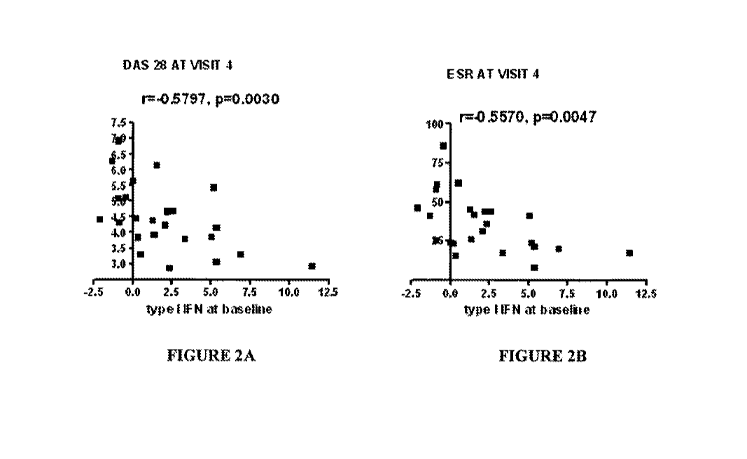

Moreover, both DAS28 (FIG. 2A) and ESR at visit 4 (FIG. 2B) (about 6.9 months) after initiation of anti-TNF therapy were significantly negatively correlated with baseline type I interferon activity.

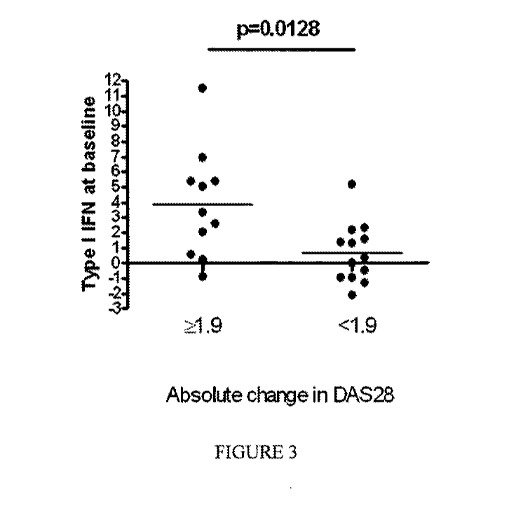

Finally, the serum plasma type I interferon level was significantly higher at baseline in those patients who demonstrated an absolute change in DAS28 score from baseline to visit 4 (about 6.9 months after initiating anti-TNF therapy) greater than or equal to 1.9 than in those patients whose DAS 28 score changed less than 1.9 over the period of anti-TNF therapy (FIG. 3).

Taken together, these data illustrate that determining type I interferon activity in a body fluid, in this case plasma, is useful for predicting therapeutic response to anti-TNF therapy, i.e., provides a basis for identifying responder patients to anti-TNF therapy. In certain embodiments, the level or activity of Type I IFN is used as a marker for determining responsiveness of RA patients to treatment with anti-TNF agents, and this effect is independent of the type of anti-TNF agent used. In additional embodiments, these methods will be useful for identifying responder patients to anti-TNF therapy, with other chronic inflammatory conditions, such as psoriatic arthritis, ankylosing spondylitis, juvenile chronic arthritis, as well as for cardiovascular disease. These methods are independent of the type of anti-TNF agent used. Additionally, the invention also provides methods for quantification of type I interferon activity as a measure of predicting responsiveness to anti-TNF therapy in patients with other inflammatory conditions that might benefit from anti-TNF therapy, including anti-TNF antibody treatment, such as Lupus and Crohn's disease. Suitable anti-TNF agents include currently available agents (e.g., anti TNF antibodies such as etanercept, infliximab or adalimumab) but also encompass additional therapeutic agents that inhibit TNF and are not limited to anti-TNF antibodies. The invention also provides methods for predicting non-responsiveness to anti-TNF therapy in patients with RA or other inflammatory conditions, permitting selection of alternative therapeutic agents other than anti-TNF agents.

### Example 2

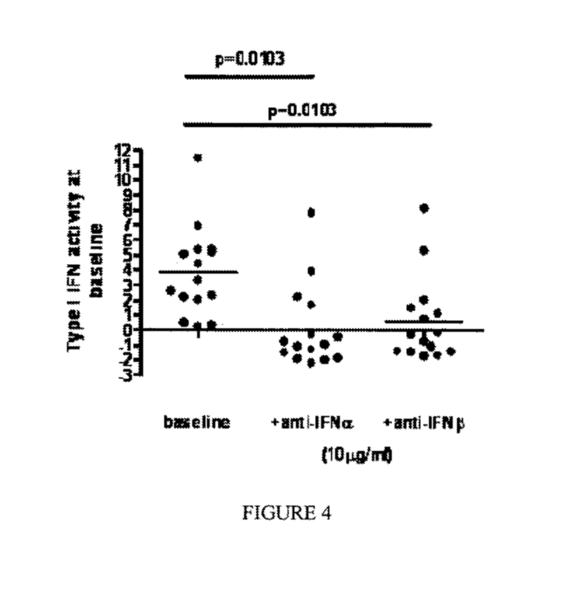

Further testing of RA plasma samples, including those assayed for Type I interferon as described in FIGS. 1-3 with specific anti-interferon antibodies, shows an interesting detection profile. In particular, the RA samples exhibit inhibition by anti-IFN-α antibodies, and also inhibition by anti-IFN-β antibodies. Combining these inhibition values and generating a ratio of the sample inhibition by anti-IFN-β compared to the inhibition conferred by anti-IFN-α has provided a useful profile for predicting response to anti-TNF therapy. As shown in FIGS. 4-6, plasma samples tested in this manner illustrated that those with a relatively higher ratio of anti-IFN-β inhibition to anti-IFN-α inhibition (presumably indicating a relatively greater proportion of IFN-β in their plasma) were the ones that had the best response to anti-TNF therapy, indicated by a lower disease activity score in these patients. It is also contemplated that an amount of IFN-β predictive for anti-TNF responders can be developed based upon a suitable sample size of results using the data of ratios of anti-IFN-β inhibition to anti-IFN-α inhibition. These methods for determining IFN-β and IFN-α activity in a serum sample provide useful diagnostic tools for identifying patients who will be responders to anti-TNF therapy, enabling efficient and cost-effective treatment of this patient population.

In addition to providing a diagnostic tool for identifying anti-TNF responder patients, detection of IFN-β in serum or plasma samples, using anti-IFN-β antibodies or any other suitable method, also provides a useful biomarker for RA generally. In particular, detection of IFN-β when used in combination with detection of IFN-α in serum or plasma samples, using anti-IFN-β and anti-IFN-α antibodies or any other suitable method including suitable antibody fragments, provides a method fox identifying patients with RA. Thus, certain embodiments of the invention provide tools for identifying RA patients by detecting IFN-β as a serum biomarker for RA.

### Example 3

### Demographics

The baseline characteristics of anti-TNF treated RA patients according to response to therapy are presented in Table 3. While no significant differences were revealed among the three groups in sex and race, medications or disease activity and duration, the group with moderate response was significantly older compared to that of the non-response group [mean (range) in years: 49(32-64) versus 38(21-55), p=0.0342].

TABLE 3
Baseline Characteristics of ANTI-TNF Treated RA Patients
Classified at Visit 4 as Good, Moderate and Non-Responders
According to EULAR Response Criteria.

| | Non Response | Moderate Response | Good Response |
|---|---|---|---|
| | (n = 7) | (n = 25) | (n = 6) |
| Age^{a} | 38 (21-55) | 49 (32-64)* | 42 (32-51) |
| Sex (F %)^{b} | 100% | 96% | 100% |
| Race (H/A/C)^{c} | 6/0/1 | 24/1/0 | 6/0/0 |
| Disease duration^{d} | 8 (0.5-30.4) | 12 (0.4-30.7) | 7 (2.7-11.6) |
| DAS28^{e} | 5.7 (4.6-7.1) | 6.4 (4.2-8.0) | 5.8 (4.5-7.4) |
| Prednisone^{f} | 57% | 28% | 50% |
| | (4 mg/d) | (2 mg/d) | (4 mg/d) |
| DMARD^{g} | | | |
| MTX | 100% | 84% | 67% |
| HCQ | 57% | 52% | 84% |
| LEF | 0% | 16% | 17% |
| SSA | 14% | 32% | 17% |
| Rheumatoid | 1121 ± 1256 | 635.2 ± 880.7 | 810 ± 1608 |
| factor levels | | | |
| (IU/ml) | | | |
| Anti-CCP levels^{h} | 88.67 ± 84.10 | 106.3 ± 65.25 | 110.5 ± 75.03 |

^{a}Mean (range) in years
^{b}F = Female
^{c}H = Hispanic, A = Asian, C = Caucasian
^{d}Mean (range) in years
^{e}Mean(range)
^{f}Percentages of patients taking prednisone (mean daily dose)
^{g}Percentages of patients taking the indicated DMARD. MTX = Methotrexate; HCQ = hydroxychloroquine; LEF = Leflunomide; SSA = sulfasalazine
^{h}data available for 20 patients
*p < 0.05 (Comparison between non response and moderate response group)-all the other comparisons between groups non significant.

In Table 4, the duration of therapy (mean±SD days) in the anti-TNF and non-anti-TNF groups throughout the follow up visits is presented.

TABLE 4
Table 4: Study participants and duration of therapy
(mean ± SD days) throughout the follow-up visits.

Months of therapy
(mean ± SD)

| | Anti-TNF | Non-anti-TNF |
|---|---|---|
| | (n = 38) | (n = 12) |
| VISIT 1 | 0 | 0 |
| VISIT 2 | 2.3 ± 1.0 | 2.3 ± 0.8 |
| VISIT 3 | 5.0 ± 2.5 | 5.0 ± 1.8 |
| VISIT 4 | 6.9 ± 2.0 | 8.8 ± 2.9 |

### Plasma Type I IFN Activity is Increased in Some RA Patients

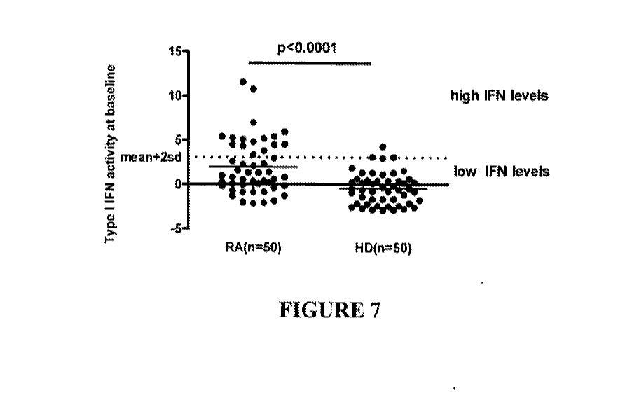

To establish the baseline level of type I IFN activity in the plasma of patients with RA, the WISH epithelial cell line assay was used to determine the capacity of plasma to induce expression of IFN-regulated genes. As shown in FIG. 7, high type I IFN levels (defined as type I IFN levels above the mean+2SD of a pool of healthy controls tested in the same assay) were detected in 32% (16/50) of all RA patients at baseline compared to 6% (3/50) of healthy controls (p<0.0001).

Type I IFN Activity at Baseline is Associated with Therapeutic Response Among RA Patients Treated with TNF Antagonists

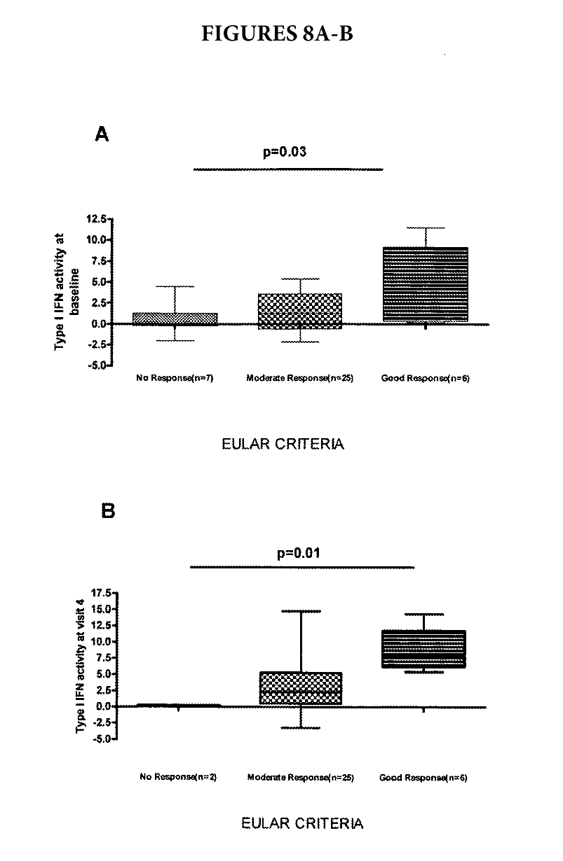

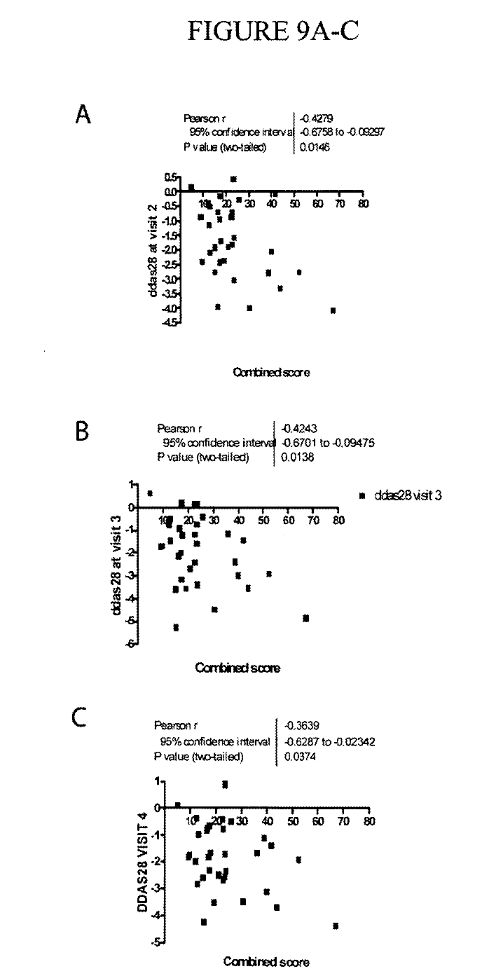

To investigate whether type I IFN plasma activity levels prior to initiation of TNF antagonist therapy is associated with response to therapy, the mean baseline plasma activity in RA patients with good, moderate, or non-response at visit 4 (6.9±0.2 months) was determined. As shown in FIG. 8A, there was a graded relationship between type I IFN activity at baseline and response to anti-TNF therapy according to EULAR criteria (mean±SD 0.60±1.20 versus 1.53±2.30 versus 4.47±4.34 in poor, moderate and good responders respectively, p=0.03). Similarly, the type I IFN plasma activity at visit 4 was significantly higher in the responders than in the non-responders (FIG. 8B; mean±SD 0.23±0.01 versus 3.34±4.11 versus 8.53±3.12, p=0.01). Of interest, there was a marginally significant correlation between type I IFN plasma activity at baseline and at visit 4 (data not shown, r=0.33, p=0.06). FIGS. 9A-C show an inverse correlation between baseline type I interferon activity and DAS28 at visits 2, 3, and 4. As early as 2.3 months after initiation of anti-TNF therapy (visit 2) lower DAS28 scores (lower disease activity) were seen in patients who had demonstrated increased plasma type I interferon activity prior to initiation of therapy. This relationship was also seen at visit 3 (approximately 5.0 months after initiating therapy) and visit 4 (approximately 6.9 months after initiating therapy). Taken together, the data suggest that measurement of type I IFN activity in RA patient plasma prior to initiation of TNF antagonist therapy serves as a predictor of response or non-response as early as 2.3 months and at 6-7 months of therapy.

TNF Antagonist Therapy is Associated with an Increase in Type I IFN Plasma Activity Over Time

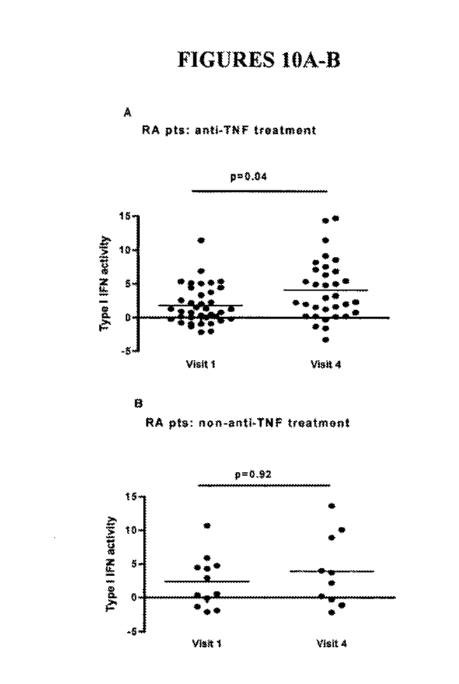

Increased serum type I IFN activity in Sjogren's syndrome patients treated for 12 weeks with etanercept, but not in those patients who received placebo in the context of a randomized controlled trial has been demonstrated (36, 38). To determine whether TNF antagonist therapy increased plasma levels of type I IFN in RA patients, IFN activity was compared at baseline and visit 4 (6.9±2.0 months) in anti-TNF treated RA patients to control RA patients who did not receive TNF antagonists (assessed at baseline and 8.8±2.9 months). A statistically significant increase in type I IFN plasma activity from baseline to visit 4 was seen in the RA group who received anti-TNF treatment (FIG. 10A). No increase in type I IFN plasma activity was observed in the control RA patients (FIG. 10B).

Increased IFNβ/α Ratio is Associated with Response to TNF Antagonist Treatment

Anti-inflammatory and anti-proliferative properties of IFNβ have been reported in the rheumatoid synovium and in animal models of inflammatory arthritis (31). Samples were analyzed to determine whether the type I IFN measured in RA plasma was predominantly IFNα or IFNβ. The percent inhibition of plasma type I IFN activity after addition of neutralizing anti-IFNα or anti-IFNβ antibody (10 μg/ml) to the plasma of those patients with elevated type I IFN activity was determined and the results described as an IFNβ/α ratio. In contrast to previous results in patients with systemic lupus erythematosus (SLE), in whom the majority of serum type I IFN activity was neutralized by anti-IFNα but not by anti-IFN antibody (37), most RA patients with elevated plasma type I IFN showed inhibition of that activity by anti-IFNβ, as well as anti-IFNα antibodies. These results suggest that in some RA patients, the capacity of their plasma to stimulate expression of type I IFN-inducible genes is attributable to IFNβ as well as IFNα.

It is likely that any type I IFN can induce a target gene to be measured in the IFN assay—IFIT1, IFI44, etc. The data described below measuring IL-1ra further support a role for IFN-β as IFN-β may be a better inducer of that protein than IFN-α.

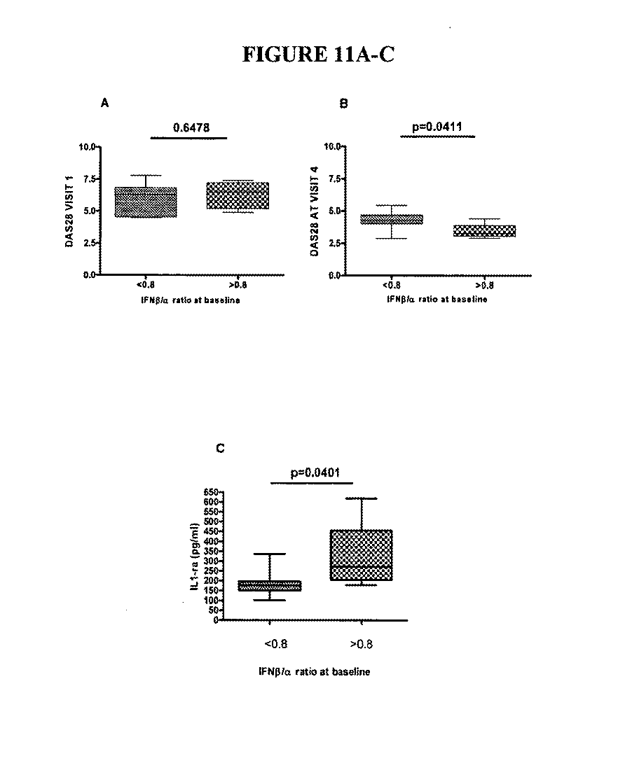

In order to elucidate further whether IFNα or IFNβ activity accounts for the association of type I IFN activity levels at baseline with response to TNF antagonist treatment, RA patients with elevated type I IFN prior to treatment with TNF antagonists were further subdivided into two groups according to their baseline IFNβ/α ratio (patients were categorized as having levels greater than or less than 0.8, the median value of the distribution). While DAS28 scores were similar in the two groups at baseline (mean±SD 5.95±1.28 for IFNβ/α ratio <0.8 versus 6.23±0.97 for IFN-β/α ratio >0.8, p=0.64), the DAS28 scores at visit 4 were significantly lower in anti-TNF treated RA patients with baseline IFNβ/α ratio >0.8 (mean±SD 4.27±0.74 for IFNβ/α ratio <0.8 versus 3.51±0.52 for IFNβ/α ratio >0.8, p=0.04), suggesting that IFNβ might represent an important contributor to the increased control of inflammation and therapeutic responses among anti-TNF treated RA individuals (FIGS. 11A and B). No differences were detected in DAS 28 score between the two groups at baseline (mean±SD 5.95±1.28 versus 6.23±0.97, p=0.64). A statistically significant decrease in DAS28 score at visit 4 was found in RA patients with baseline ratio IFNβ/α >0.8 compared to those with IFNβ/α <0.8 (mean±SD 3.51±0.52 versus 4.27±0.74 p=0.04). No other differences in demographic variables or treatment modalities were detected between the two groups (Table 5).

TABLE 5
Baseline Characteristics and Therapeutic Response of Anti-
TNF Treated RA Patients Classified by the IFNβ/α ratio.

| | IFNβ/α < 0.8 | IFNβ/α > 0.8 |
|---|---|---|
| | (n = 8) | (n = 7) |
| DAS28 Baseline | 5.95 ± 1.28 | 6.23 ± 0.97 |
| DAS28 Visit 4 | 4.27 ± 0.74 | 3.51 ± 0.52^{a} |
| Age^{b} | 48 (36-60) | 47 (32-60) |
| Sex (F %)^{c} | 87.5% | 100% |
| Race (H/A)^{d} | 1/7 | 0/8 |
| Disease duration^{e} | 8.9 (0.4-22) | 6.9 (2.7-12.6) |
| Prednisone^{f} | 37.5% | 28.5% |
| | (3.125 mg/d) | (25 mg/d) |
| DMARD^{g} | | |
| MTX | 87.5% | 71.4% |
| HCQ | 62.5% | 42.8% |
| LEF | 12.5% | 28.5% |
| SSA | 37.5% | 28.5% |

^{a}P = 0.04 for IFN-β/α >0.8 compared with IFN-β/α <0.8 at visit 4.
^{b}Mean (range) in years
^{c}F = Female
^{d}H = Hispanic, A = Asian
^{e}Mean (range) in years
^{f}Percentages of patients taking prednisone (mean daily dose)
^{g}Percentages of patients taking the indicated DMARD. MTX = Methotrexate; HCQ = hydroxychloroquine; LEF = Leflunomide; SSA = sulfasalazine

IL-1Ra is Increased in RA Patients with a High Plasma IFNβ/α Ratio.

Previous in vitro studies have demonstrated induction of IL-1ra by IFNβ in PBMC and synoviocytes (39, 40). IL-1ra may represent an IFNβ-induced mediator that contributes to the superior therapeutic response to TNF antagonist therapy. Thus, IL-1ra levels were measured in RA patient plasma samples. As shown in FIG. 11C, significantly higher levels of IL-1ra were present in the RA patients with baseline IFNβ/α ratio >0.8 compared to those with baseline IFNβ/α ratio <0.8 (mean±SD 322.6±161.0 versus 185.8±68.72, p=0.04).

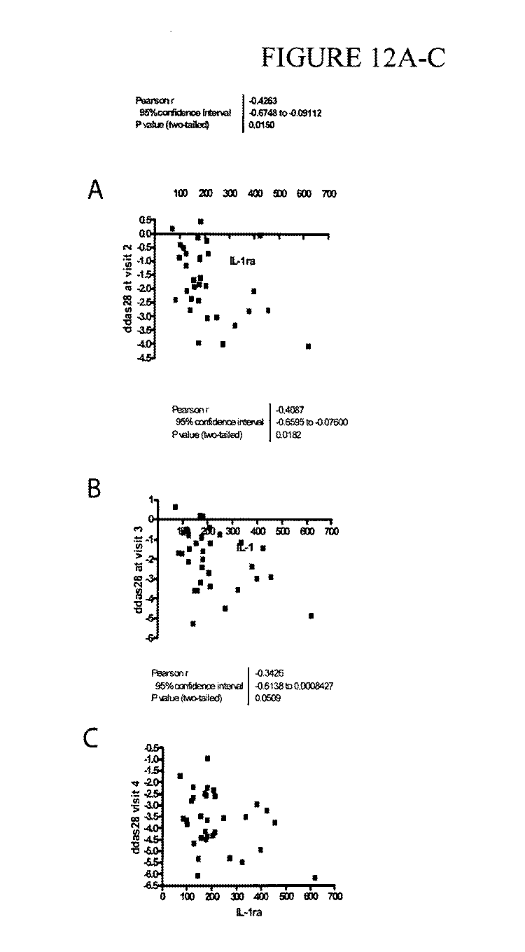

FIGS. 12A-C show an inverse correlation between baseline plasma IL-1ra and DAS28 score at visit 2, 3 and 4. Data from visit 4 were only borderline significant, but visits 2 and 3 showed a significant inverse correlation. These data indicate that IL-1ra, a protein induced by IFN-β, could also be used as a predictive biomarker of response to anti-TNF therapy.

### DISCUSSION

TNF antagonists have provided important treatment options for RA and other inflammatory conditions over recent years. However, the responsiveness to treatment is highly variable among individuals. Interestingly, this lack of response seems to be a stable trait over time, with further infusions adding very limited benefit to primary non-responders, suggesting that genetically determined factors affecting either the pharmacokinetics or the pharmacodynamics of these agents may account for the differential responses among individuals (21). Another possible contributor to the highly heterogeneous response to treatment is the diversity and complexity of pathogenic mechanisms. Recent gene expression data from PBMC and synovial tissues from patients with RA support the heterogeneous nature of the disease and highlight the role of type I IFN in a subset of patients (26, 27, 41). While the factors that contribute to type I IFN activity and IFN pathway activation in the setting of RA have not been determined, previous data have demonstrated that IFNβ is present in the rheumatoid synovium of some patients and is associated with control of inflammatory responses (31, 42, 43).

In view of the high cost of treatment and the potentially serious adverse effects, identification of predictors of responses to anti-TNF agents would be extremely useful in clinical practice. With the availability of additional new therapeutic options, patients predicted to be TNF antagonist non-responders might be more successfully treated with other agents, leading to earlier therapeutic responses. Type I IFN activity in some patients with RA contributes to better inflammatory control and serves as a predictor of response to therapy.

In the data described herein, including FIGS. 1-12, a number of methods, including using a sensitive reporter cell assay, identified RA patients characterized by increased plasma type I IFN levels compared to a pool of healthy controls tested in the same assay. Importantly, higher levels of type I IFN activity at baseline and at the end of follow up (visit 4) were associated with better treatment outcomes as defined by EULAR criteria. Thus, RA patients with increased plasma type I IFN levels compared to a pool of healthy controls tested in the same assay, were classified as responders to anti-TNF agents.

In contrast to previously reported data in SLE, where IFNα appeared to be a major contributor to the type I IFN activity detected in lupus plasma (37), inhibition experiments using monoclonal anti-IFNα and anti-IFNβ antibodies revealed that both IFNα and IFNβ contribute to type I IFN activity detected in RA plasma.

In order to explore further which component of type I IFN was associated with better outcomes, an IFNβ/α ratio was calculated based on the % inhibition of type I IFN activity by anti-IFNβ and anti-IFNα antibodies. A higher IFNβ/α ratio was found to be associated with better responses, indicating that IFNβ rather than IFNα is likely to be a significant contributor to control of inflammation and better disease outcomes in the setting of RA. Of interest, baseline plasma samples with a high IFNβ/α ratio contained significantly higher levels of IL-1ra, an anti-inflammatory cytokine that has been shown to be induced by IFNβ. While IFNβ is likely to have direct effects on synovial tissue that contribute to control of inflammation and induces a broad spectrum of IFN-inducible genes, the association of high IFNβ activity with good clinical outcomes in response to TNF antagonist therapy, as well as increased plasma IL-1ra suggests at least one mechanism for the responses observed.

The anti-inflammatory role of IFNβ has been previously suggested based on in vitro experiments and studies of murine models of inflammatory arthritis. IFNβ is a type I IFN with pleiotropic immunomodulatory actions including down regulation of the proinflammatory cytokines IL-1β and TNF-α and enhancement of anti-inflammatory cytokines IL-1ra, soluble TNF receptor II, IL-10 and transforming growth factor β (39, 40, 44, 45). It has also been shown to mediate inhibition of HLA class 11 on activated PBMC (45), inhibition of T-cell activation (46) and down regulation of adhesion molecules (47). In collagen-induced arthritis models, intraperitoneal injection of IFNβ has resulted in reduction of disease activity, inhibition of cartilage and bone destruction through significant decrease of TNF and IL-6 expression and enhancement of IL-10 responses at the site of inflammation (48).

The results provided in the Examples described herein illustrate that type I IFN activity at baseline in RA patients with poor, moderate and good responses at visit 4 (6.9±2.0 months) showed a significant graded increase, with high IFN activity associated with a good response among anti-TNF treated patients. At visit 4, disease activity based on DAS28 score was significantly lower in patients with a baseline plasma IFNβ/α ratio >0.8, indicating elevated plasma IFNβ. Consistent with the capacity of IFNβ to induce IL-1 receptor antagonist (IL-1ra), baseline plasma samples from patients with elevated IFNβ/α ratios contained significantly higher levels of IL-1ra.

These data also illustrated that baseline elevated IFNβ activity, rather than IFNα, contributes to the improved responses as indicated by its association with elevated plasma IL-1ra. Taken together, the data support the utility of measurement of type I IFN activity, and more specifically IFNβ activity, as a predictor of response or non-response to TNF antagonist therapy. Elevated plasma type I IFN activity and IFNβ/α ratio, as well as plasma IL-1ra, measured prior to initiation of TNF antagonist therapy were associated with a good therapeutic response in RA patients, identifying those mediators as candidate biomarkers for response to TNF blockade in RA.

### REFERENCES

1. Maini R N, Breedveld F C, Kalden J R, Smolen J S, Davis D, Macfarlane J D, et al. Therapeutic efficacy of multiple intravenous infusions of anti-tumor necrosis factor alpha monoclonal antibody combined with low-dose weekly methotrexate in rheumatoid arthritis. Arthritis Rheum 1998; 41(9):1552-63.
2. Maini R, St Clair E W, Breedveld F. Furst D, Kalden J, Weisman M, et al. Infliximab (chimeric anti-tumour necrosis factor alpha monoclonal antibody) versus placebo in rheumatoid arthritis patients receiving concomitant methotrexate: a randomised phase III trial. ATTRACT Study Group. Lancet 1999; 354(9194):1932-9.
3. Lipsky P E, van der Heijde D M, St Clair E W, Furst D E, Breedveld F C, Kalden J R, et al. Infliximab and methotrexate in the treatment of rheumatoid arthritis. Anti-Tumor Necrosis Factor Trial in Rheumatoid Arthritis with Concomitant Therapy Study Group. N Engl J Med 2000; 343(22):1594-602.
4. Kavanaugh A, St Clair E W, McCune W J, Braakman T, Lipsky P. Chimeric anti-tumor necrosis factor-alpha monoclonal antibody treatment of patients with rheumatoid arthritis receiving methotrexate therapy. J Rheumatol 2000; 27(4):841-50.
5. Moreland L W, Schiff M K Baumgartner S W, Tindall E A, Fleischmann R M, Bulpitt K J, et al. Etanercept therapy in rheumatoid arthritis. A randomized, controlled trial. Ann Intern Med 1999; 130(6):478-86.
6. Weinblatt M E, Kremer J M, Bankhurst A D, Bulpitt K J, Fleischmann R M, Fox R I, et al. A trial of etanercept, a recombinant tumor necrosis factor receptor:Fc fusion protein, in patients with rheumatoid arthritis receiving methotrexate. N Engl J Med 1999; 340(4):253-9.
7. Weinblatt M E, Keystone E C, Furst D E, Moreland L W, Weisman M H, Birbara C A, et al. Adalimumab, a fully human anti-tumor necrosis factor alpha monoclonal antibody, for the treatment of rheumatoid arthritis in patients taking concomitant methotrexate: the ARMADA trial. Arthritis Rheum 2003; 48(1):35-45.
8. Klareskog L, van der Heijde D, de Jager J P, Gough A, Kalden J, Malaise M, et al. Therapeutic effect of the combination of etanercept and methotrexate compared with each treatment alone in patients with rheumatoid arthritis: double-blind randomised controlled trial. Lancet 2004; 363(9410):675-81.
9. Keystone E C, Schiff M H, Kremer J M, Kafka 5, Lovy M, DeVries T, et al. Once-weekly administration of 50 mg etanercept in patients with active rheumatoid arthritis: results of a multicenter, randomized, double-blind, placebo-controlled trial. Arthritis Rheum 2004; 50(2):353-63.
10. Keystone E C, Kavanaugh A F, Sharp J T, Tannenbaum H, Hua Y, Teoh L S, et al. Radiographic, clinical, and functional outcomes of treatment with adalimumab (a human anti-tumor necrosis factor monoclonal antibody) in patients with active rheumatoid arthritis receiving concomitant methotrexate therapy: a randomized, placebo-controlled, 52-week trial. Arthritis Rheum 2004; 50(5):1400-11.
11. Breedveld F C, Weisman M H, Kavanaugh A F, Cohen S B, Pavelka K, van Vollenhoven R, et al. The PREMIER study: A multicenter, randomized, double-blind clinical trial of combination therapy with adalimumab plus methotrexate versus methotrexate alone or adalimumab alone in patients with early, aggressive rheumatoid arthritis who had not had previous methotrexate treatment. Arthritis Rheum 2006; 54(1):26-37.
12. St Clair E W, van der Heijde D M, Smolen J S, Maini R N, Bathon J M, Emery P. et al. Combination of infliximab and methotrexate therapy for early rheumatoid arthritis: a randomized, controlled trial. Arthritis Rheum 2004; 50(11):3432-43.
13. Hyrich K L, Watson K D, Silman A J, Symmons D P. Predictors of response to anti-TNF-alpha therapy among patients with rheumatoid arthritis: results from the British Society for Rheumatology Biologics Register. Rheumatology (Oxford) 2006; 45(12):1558-65.
14. Mancarella L, Bobbio-Pallavicini F, Ceccarelli F, Falappone P C, Ferrante A, Malesci D, et al. Good clinical response, remission, and predictors of remission in rheumatoid arthritis patients treated with tumor necrosis factor-alpha blockers: the GISEA study. J Rheumatol 2007; 34(8):1670-3.
15. Braun-Moscovici Y, Markovits D, Zinder O, Schapira D, Rozin A, Ehrenburg M, et al. Anti-cyclic citrullinated protein antibodies as a predictor of response to anti-tumor necrosis factor-alpha therapy in patients with rheumatoid arthritis. J Rheumatol 2006; 33(3):497-500.
16, Potter C, Hyrich K L, Tracey A, Lunt M, Plant D, Symmons D P, et al. Association of R F and anti-CCP positivity, but not carriage of shared epitope or PTPN22 susceptibility variants, with anti-TNF response in R A. Ann Rheum Dis 2008.
17, van der Pouw Kraan T C, Wijbrandts C A, van Baarsen L G, Rustenburg F, Baggen J M, Verweij C L, et al. Responsiveness to anti-TNF{alpha} therapy is related to pre-treatment tissue inflammation levels in Rheumatoid Arthritis patients. Ann Rheum Dis 2007.
18. Wijbrandts C A, Dijkgraaf M G, Kraan M C, Vinkenoog M, Smeets T J, Dinant H, et al. The clinical response to infliximab in rheumatoid arthritis is in part dependent on pre-treatment TNF{alpha} expression in the synovium. Ann Rheum Dis 2007,
19. Legurre T, Gauthier-Jauneau A C, Bansard C, Derambure C, Hiron M, Vittecoq O, et al. Gene profiling in white blood cells predicts infliximab responsiveness in rheumatoid arthritis. Arthritis Res Ther 2006; 8(4):R105.
20. Koczan D, Drynda S, Hecker M, Drynda A, Guthke R, Kekow J, et al. Molecular discrimination of responders and nonresponders to anti-TNFalpha therapy in rheumatoid arthritis by etanercept. Arthritis Res Ther 2008; 10(3):R50.
21. Mascheretti S, Schreiber S. The role of pharmacogenomics in the prediction of efficacy of anti-TNF therapy in patients with Crohn's disease. Pharmacogenomics 2004; 5(5):479-86.
22. Maxwell J R, Potter C, Hyrich K L, Barton A, Worthington J, Isaacs J D, et al. Association of the tumour necrosis factor-308 variant with differential response to anti-TNF agents in the treatment of rheumatoid arthritis. Hum Mol Genet 2008; 17(22):3532-8.
23. Mugnier B, Balandraud N, Darque A, Roudier C, Roudier J, Reviron D. Polymorphism at position −308 of the tumor necrosis factor alpha gene influences outcome of infliximab therapy in rheumatoid arthritis. Arthritis Rheum 2003; 48(7):1849-52.
24. Liu C, Batliwalla F, Li W, Lee A, Roubenoff R, Beckman E, et al. Genome-wide association scan identifies candidate polymorphisms associated with differential response to anti-TNF treatment in rheumatoid arthritis. Mol Med 2008; 14(9-10):575-81.
25. Edwards C J, Feldman J L, Beech J, Shields K M, Stover J A, Trepicchio W L, et al. Molecular profile of peripheral blood mononuclear cells from patients with rheumatoid arthritis. Mol Med 2007; 13(1-2):40-58.
26. Batliwalla F M, Baechler E C, Xiao X, Li W, Balasubramanian S, Khalili H, et al. Peripheral blood gene expression profiling in rheumatoid arthritis. Genes Immun 2005; 6(5):388-97.
27. Bovin L F, Rieneck K, Workman C, Nielsen H, Sorensen S F, Skjodt H, et al. Blood cell gene expression profiling in rheumatoid arthritis. Discriminative genes and effect of rheumatoid factor. Immunol Lett 2004; 93(2-3):217-26.
28. van der Pouw Kraan T C, van Gaalen F A, Kasperkovitz P V, Verbeet N L, Smeets T J, Kraan M C, et al. Rheumatoid arthritis is a heterogeneous disease: evidence for differences in the activation of the STAT-1 pathway between rheumatoid tissues. Arthritis Rheum 2003; 48(8):2132-45.
29. Hueber W, Kidd B A, Tomooka B H, Lee B J, Bruce B, Fries J F, et al. Antigen microarray profiling of autoantibodies in rheumatoid arthritis. Arthritis Rheum 2005; 52(9):2645-55.
30. van der Pouw Kraan T C, van Baarsen L G, Wijbrandts C A, Voskuyl A E, Rustenburg F, Baggen J M, et al. Expression of a pathogen-response program in peripheral blood cells defines a subgroup of rheumatoid arthritis patients. Genes Immun 20089(1):16-22.
31. Tak P P. IFN-beta in rheumatoid arthritis. Front Biosci 2004; 9:3242-7.
32. La D T, Collins C E, Yang H T, Migone T S, Stohl W. B lymphocyte stimulator expression in rheumatoid arthritis patients treated with tumor necrosis factor {alpha} antagonists: differential effects between good and poor clinical responders. Ann Rheum Dis 2007.
33. van Gestel A M, Haagsma C J, van Rid P L. Validation of rheumatoid arthritis improvement criteria that include simplified joint counts. Arthritis Rheum 1998; 41(10):1845-50.
34. Fransen J, van Riel P L. The Disease Activity Score and the EULAR response criteria. Clin Exp Rheumatol 2005; 23(5 Suppl 39):S93-9.
35. Niewold T B, Hua J, Lehman Ti, Harley J B, Crow M K. High serum IFN-alpha activity is a heritable risk factor for systemic lupus erythematosus. Genes Immun 2007; 8(6):492-502.
36. Mavragani C P, Niewold T B, Moutsopoulos N M, Pillemer S R, Wahl S M, Crow M K. Augmented interferon-alpha pathway activation in patients with Sjogren's syndrome treated with etanercept. Arthritis Rheum 2007; 56(12):3995-4004.
37. Hua J, Kirou K, Lee C, Crow M K. Functional assay of type I interferon in systemic lupus erythematosus plasma and association with anti-RNA binding protein autoantibodies. Arthritis Rheum 2006; 54(6): 1906-16.
38. Sankar V, Brennan M T, Kok M R, Leakan R A, Smith J A, Manny J, et al. Etanercept in Sjogren's syndrome: a twelve-week randomized, double-blind, placebo-controlled pilot clinical trial. Arthritis Rheum 2004; 50(7):2240-5.
39. Coclet-Ninin J, Dayer J M, Burger D. Interferon-beta not only inhibits interleukin-1beta and tumor necrosis factor-alpha but stimulates interleukin-1 receptor antagonist production in human peripheral blood mononuclear cells. Eur Cytokine Netw 1997; 8(4):345-9.
40. Palmer G, Mezin F, Juge-Aubry C E, Plater-Zyberk C, Gabay C, Guerre P A. Interferon beta stimulates interleukin 1 receptor antagonist production in human articular chondrocytes and synovial fibroblasts. Ann Rheum Dis 2004; 63(1):43-9.
41. van der Pouw Kraan T C, Wijbrandts C A, van Baarsen L G, Voskuyl A E, Rustenburg F, Baggen J M, et al. Rheumatoid arthritis subtypes identified by genomic profiling of peripheral blood cells: assignment of a type I interferon signature in a subpopulation of patients. Ann Rheum Dis 2007; 66(8):1008-14.
42. van Holten J, Smeets T J, Blankert P. Tak P P. Expression of interferon beta in synovial tissue from patients with rheumatoid arthritis: comparison with patients with osteoarthritis and reactive arthritis. Ann Rheum Dis 2005; 64(12):1780-2.
43. Treschow A P, Teige I, Nandakumar K S, Holmdahl R, Issazadeh-Navikas S. Stromal cells and osteoclasts are responsible for exacerbated collagen-induced arthritis in interferon-beta-deficient mice. Arthritis Rheum 2005; 52(12):3739-48.
44. van Holten J, Plater-Zyberk C, Tak P P. Interferon-beta for treatment of rheumatoid arthritis? Arthritis Res 2002; 4(6):346-52.
45. Yong V W, Chabot 5, Stuve O, Williams G. Interferon beta in the treatment of multiple sclerosis: mechanisms of action. Neurology 1998; 51(3):682-9.
46. Noronha A, Toscas A, Jensen M A. Interferon beta decreases T cell activation and interferon gamma production in multiple sclerosis. J Neuroimmunol 1993; 46(1-2):145-53,
47. Calabresi P A, Pelfrey C M, Tranquill L R, Maloni H, McFarland H F. VLA-4 expression on peripheral blood lymphocytes is downregulated after treatment of multiple sclerosis with interferon beta. Neurology 1997; 49(4):1111-6.
48. van Holten J, Reedquist K, Sattonet-Roche P, Smeets T J, Plater-Zyberk C, Vervoordeldonk M J, et al. Treatment with recombinant interferon-beta reduces inflammation and slows cartilage destruction in the collagen-induced arthritis model of rheumatoid arthritis. Arthritis Res Ther 2004; 6(3):R239-49.
49, Tak P P, Hart B A, Kraan M C, Jonker M, Smeets T J, Breedveld F C. The effects of interferon beta treatment on arthritis. Rheumatology (Oxford) 1999; 38(4):362-9.
50. Smeets T J, Dayer J M, Kraan M C, Versendaal J, Chicheportiche R, Breedveld F C, et al. The effects of interferon-beta treatment of synovial inflammation and expression of metalloproteinases in patients with rheumatoid arthritis. Arthritis Rheum 2000; 43(2):270-4.
51. Adriaansen J, Kuhlman R R, van Holten J, Kaynor C, Vervoordeldonk M J, Tak P P. Intraarticular interferon-beta gene therapy ameliorates adjuvant arthritis in rats. Hum Gene Ther 2006; 17(10):985-96.
52. Adriaansen J, Fallaux F J, de Cortie C J, Vervoordeldonk M J, Tak P P. Local delivery of beta interferon using an adeno-associated virus type 5 effectively inhibits adjuvant arthritis in rats. J Gen Virol 2007; 88(Pt 6):1717-21.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

While the compositions and methods of this invention have been described in terms of specific embodiments, it will be apparent to those of skill in the art that variations may be applied to the compositions and methods and in the steps or in the sequence of steps of the method described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

Patents, patent applications, publications, product descriptions, and protocols are cited throughout this application, the disclosures of which are incorporated herein by reference in their entireties for all purposes.

1. A method for identifying a patient as a candidate for an anti-tumor necrosis factor (TNF) therapy comprising:
(a) contacting type I interferon responsive cells in vitro with a body fluid sample obtained from the patient before undergoing anti-TNF therapy, wherein the interferon responsive cells are not the patient's cells;
(b) detecting expression levels of one or more interferon-inducible genes (IFIGs) by the type I interferon responsive cells contacted with the body fluid sample;
(c) determining from the detected expression levels, an interferon score indicative of type I interferon activity in the body fluid sample;
(d) identifying the patient as a candidate for anti-TNF therapy when the determined interferon score indicates elevated type I interferon activity compared to type I interferon activity obtained from cells that have been exposed or contacted with a control sample.

2. The method of claim 1, wherein the type I interferon responsive cells are selected from the group consisting of A-549 cells, AG1732 cells, HeLa cells, HepG2 cells, Hep-2 cells, Huh-7 cells, G-361 cells and WISH cells.

3. The method of claim 2, wherein the type I interferon responsive cells are WISH cells.

4. The method of claim 1, wherein the detecting is carried out using real-time quantitative PCR or a luciferase reporter assay.

5. The method of claim 1, wherein an interferon score compares interferon-alpha (IFNα) activity in the body fluid sample to interferon-beta (IFNβ) activity in the body fluid sample.

6. The method of claim 1, wherein the body fluid sample is plasma or serum.

7. A method for identifying a patient as a candidate for an anti-tumor necrosis factor (TNF) therapy comprising:
(a) contacting type I interferon responsive cells in vitro with a body fluid sample obtained from the patient before undergoing the anti-TNF therapy, wherein the type I interferon responsive cells are not the patient's cells;
(b) detecting expression levels of one or more type I interferon-inducible gene genes (IFIGs) by the type I interferon responsive cells contacted with the body fluid sample;
(c) comparing the detected expression levels with expression levels of the one or more IFIGs by the type I interferon responsive cells contacted with a control sample, and
(d) identifying the patient as a candidate for anti-TNF therapy when the detected expression levels are increased as compared with the expression levels by type I interferon responsive cells contacted with the control sample.

8. The method of claim 7, wherein the type I interferon responsive cells are selected from the group consisting of A-549 cells, AG1732 cells, HeLa cells, HepG2 cells, Hep-2 cells, Huh-7 cells, G-361 cells and WISH cells.

9. The method of claim 8, wherein the type I interferon responsive cells are WISH cells.

10. The method of claim 7, wherein the detecting is carried out using real-time quantitative PCR or a luciferase reporter assay.

11. The method of claim 7, wherein the body fluid sample is plasma or serum.

12. A method for diagnosing responsiveness to an inhibitor of tumor necrosis factor (TNF) in a patient with a chronic inflammatory disease comprising determining the activity of interferon-beta (IFN-β) compared to the activity of interferon alpha (IFN-α) in a body fluid sample from the patient.

13. The method of claim 12, wherein the determining is carried out using one or more of the methods selected from the group consisting of real-time quantitative PCR, luciferase assay and ELISA.

14. The method of claim 12, wherein the body fluid sample is plasma or serum.

15. A method for diagnosing a rheumatoid arthritis patient comprising determining the activity of interferon-beta (IFN-β) in a body fluid sample in a patient.

16. The method of claim 15, wherein determining is carried out using one or more of the methods selected from the group consisting of real-time quantitative PCR, luciferase assay and ELISA.

17. The method of claim 16, wherein the body fluid sample is plasma or serum.

18. A method for identifying a patient as a candidate for anti-tumor necrosis factor (TNF) therapy, which method comprises determining type I interferon activity in a body fluid sample obtained from the patient before undergoing the anti-TNF therapy; and identifying the patient as a candidate for anti-TNF therapy when the determined type I interferon activity is elevated relative to a control body fluid sample.

19. The method of claim 18, wherein the determining is carried out using real-time quantitative PCR, a luciferase assay, or ELISA.

20. The method of claim 18, wherein the body fluid sample is plasma or serum.

21. A method for diagnosing responsiveness to an inhibitor of tumor necrosis factor (TNF) in a patient with rheumatoid arthritis or chronic inflammatory disease comprising determining expression of at least one target protein responsive to type I interferon, in a body fluid sample of the patient.

22. The method of claim 21, wherein the determining is carried out by measuring increased expression of at least one interferon-beta induced gene using one or more of the methods selected from the group consisting of real-time quantitative PCR, luciferase assay and ELISA.

23. The method of claim 21, wherein the protein is interleukin-1 receptor antagonist (IL-1ra) or a panel of one or more interferon-beta responsive proteins.

24. The method of claim 5, wherein the patient is identified as a candidate for anti-TNF therapy when the ratio of IFNβ activity to IFNα activity in the body fluid sample is greater than 0.8.


SEQUENCE LISTING (8 sequences)

SEQ ID NO: 1 - DNA, 24 bp, Artificial Sequence
  primer
ctccttgggttcgtctataaattg

SEQ ID NO: 2 - DNA, 19 bp, Artificial Sequence
  primer
agtcagcagccagtctcag

SEQ ID NO: 3 - DNA, 20 bp, Artificial Sequence
  primer
cttccatctgactcaggttt

SEQ ID NO: 4 - DNA, 21 bp, Artificial Sequence
  primer
tgcttctgacggtatgtatta

SEQ ID NO: 5 - DNA, 19 bp, Artificial Sequence
  primer
taccaggactacgagattg

SEQ ID NO: 6 - DNA, 19 bp, Artificial Sequence
  primer
tgccaggaaggtctattag

SEQ ID NO: 7 - DNA, 19 bp, Artificial Sequence
  primer
caacggatttggtcgtatt

SEQ ID NO: 8 - DNA, 20 bp, Artificial Sequence
  primer
gatggcaacaatatccactt